(19) United States
(12) Patent Application Publication
(10) Pub. No.: US 2026/0292421 A1
(43) Pub. Date: Sep. 24, 2026

(54) PREDICTIVE TECHNIQUES FOR SENSORY AIDS

(71) Applicant: COCHLEAR LIMITED, Macquarie University, NSW (AU)

(72) Inventors: Christopher BENNETT, Macquarie University (AU); Griet GOOVAERTS, Macquarie University (AU); Ryan Orin MELMAN, Macquarie University (AU); Stephen KLINEBERG, Macquarie University (AU); Ben FUTERAN, Macquarie University (AU); Malcom RIDDELL, Macquarie University (AU)

(21) Appl. No.: 19/476,339

(22) PCT Filed: Apr. 17, 2024
(86) PCT No.: PCT/IB2024/053758

§ 371 Date: Oct. 17, 2025

Related U.S. Application Data
(60) Provisional application No. 63/460,864, filed on Apr. 20, 2023.

(51) Int. Cl. H04R 25/00; A61B 5/00; A61B 5/12

(52) U.S. Cl. CPC H04R 25/70; A61B 5/125; A61B 5/4851; A61B 5/7267; H04R 25/606

(57) ABSTRACT

A method including obtaining data relating to at least demographic data and sensory performance of a human, analyzing the data based on the obtained data using a statistical model, a probabilistic model and/or a model based on results from or that is a product of machine learning to develop output, wherein at least one of the output is a prediction of results relating to application of a sensory supplement device to the human or the method further includes developing a prediction of results relating to application of a sensory supplement device to the human based on the output.

### CROSS-REFERENCE TO RELATED APPLICATIONS

This application claims priority to U.S. Provisional Application No. 63/460,864, entitled PREDICTIVE TECHNIQUES FOR SENSORY AIDS, filed on Apr. 20, 2023, naming Christopher BENNETT as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a method, comprising: obtaining data relating to at least demographic data and sensory performance of a human; analyzing the data based on the obtained data using a statistical model, a probabilistic model and/or a model based on results from or that is a product of machine learning to develop output, wherein at least one of: the output is a prediction of results relating to application of a sensory supplement device to the human; or the method further includes developing a prediction of results relating to application of a sensory supplement device to the human based on the output.

In an exemplary embodiment, there is a method, comprising: obtaining data pertaining to a person with a medical condition; and analyzing the obtained data using a computer chip or a logic circuit or electronics or software to develop data regarding effects of treatment scenarios of the person if that person engages in the treatment, wherein the a computer chip or a logic circuit or electronics or software is based on a statistically significant population of persons who were afflicted with the medical condition and who also engaged in the treatment.

In an exemplary embodiment, there is a method, comprising: obtaining (i) habilitation and/or rehabilitation data and/or sensory health data and (ii) demographic data for a statistically significant number of sensory impaired individuals; and analyzing the obtained data to develop a predictive algorithm for human sensory performance based on the results of the analysis, wherein the predictive algorithm predicts performance based on input specific to a sensory impaired person who is not one of the individuals.

In an exemplary embodiment, there is a device, comprising: an input suite configured to receive input relating to a sensory impaired person and electronics configured to analyze data based on input into the input suite, wherein the electronics includes at least one of: a prediction model that is used by the device to automatically predict performance capabilities of the sensory impaired person after receiving a sensory aid and using such for a given period of time based on the input into the input suite; or a cohort comparator that is used by the device to automatically identify a cohort of people with statistically significantly similar and/or the same pre-sensory aid use factors as the sensory impaired person.

In an exemplary embodiment, there is a system, comprising: an input subsystem configured to receive input regarding information about a hearing impaired person; an output subsystem; and an artificial intelligence subsystem interposed between the input subsystem and the output subsystem, wherein the system is configured to predict, with the use of the artificial intelligence system, hearing performance of the hearing impaired person resulting from use of a hearing device and/or resulting from lack of use of the hearing device based on the input into the input subsystem, and wherein the system.

In an exemplary embodiment, there is a method, comprising: obtaining assess to a database having data based on hearing habilitation and/or rehabilitation data and demographic data for a statistically significant number of individuals; obtaining data on a hearing impaired person who is not one of the individuals; querying the database based on the obtained data for the hearing impaired person to automatically identify a cohort of similar individuals to the hearing impaired person from among a statistically significant number of individuals; and retrieving post hearing device usage clinical measures and/or functional outcomes for the individuals corresponding to the identified cohort. And in an embodiment, there is a non-transitory computer readable medium having recorded thereon, a computer program for executing at least a portion of a method, the computer program including:

code for obtaining (i) habilitation and/or rehabilitation data and/or sensory health data and (ii) demographic data for a statistically significant number of sensory impaired individuals; and code for analyzing the obtained data to develop a predictive algorithm for human sensory performance based on the results of the analysis, wherein the predictive algorithm predicts performance based on input specific to a sensory impaired person who is not one of the individuals.

### BRIEF DESCRIPTION OF THE DRAWINGS

Embodiments are described below with reference to the attached drawings, in which:

FIG. 1 is a perspective view of an exemplary hearing prosthesis;

FIG. 2 presents a functional block diagram of an exemplary cochlear implant;

FIG. 3A and FIGS. 3B and 3C present exemplary systems of communication between devices;

FIG. 4 presents an exemplary retinal prosthesis;

FIG. 5 presents an exemplary vestibular implant;

FIGS. 6-7, 8, 17, 19, and 20 show flowcharts for exemplary methods;

FIG. 7A presents an exemplary system diagram;

FIGS. 9-12, 18, 21, and 22 present exemplary system diagrams; and

FIGS. 13-16 present exemplary windows for the transfer of information.

### DETAILED DESCRIPTION

Merely for ease of description, the techniques presented herein are described herein with reference by way of background to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used to determine the viability of various types of prostheses, such as, for example, a vestibular implant and/or a retinal implant, with respect to a particular human being. And with regard to the latter, the techniques presented herein are also described with reference by way of background to another illustrative medical device, namely a retinal implant. The techniques presented herein are also applicable to the technology of vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

Also, embodiments are directed to other types of hearing prostheses, such as middle ear implants, bone conduction devices (active transcutaneous, passive transcutaneous, percutaneous), and conventional hearing aids. Thus, embodiments are directed to devices that include implantable portions and embodiments that do not include implantable portions.

Any reference to one of the above-noted sensory prostheses corresponds to an alternate disclosure using one of the other above-noted sensory prostheses unless otherwise noted, providing that the art enables such.

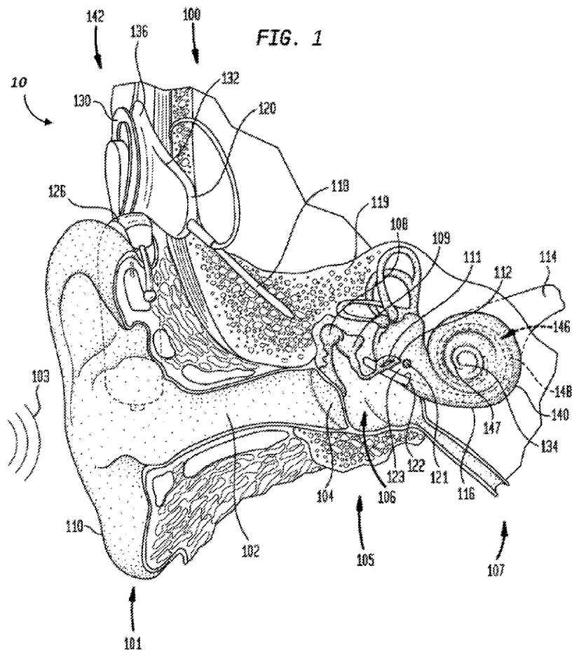

FIG. 1 is a perspective view of a cochlear implant, referred to as cochlear implant 100, implanted in a recipient, to which some embodiments detailed herein and/or variations thereof are applicable. Particularly, as will be detailed below, there are aspects of a cochlear implant that are utilized with respect to a vestibular implant, and thus there is utility in describing features of the cochlear implant for purposes of understanding a vestibular implant. The cochlear implant 100 is part of a system 10 that can include external components in some embodiments, as will be detailed below. Additionally, it is noted that the teachings detailed herein are also applicable to other types of hearing prostheses, such as, by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). Again, it is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Thus, as seen above, one variety of implanted devices depends on an external component to provide certain functionality and/or power. For example, the recipient of the implanted device can wear an external component that provides power and/or data (e.g., a signal representative of sound) to the implanted portion that allow the implanted device to function. In particular, the implanted device can lack a battery and can instead be totally dependent on an external power source providing continuous power for the implanted device to function. Although the external power source can continuously provide power, characteristics of the provided power need not be constant and may fluctuate. Additionally, where the implanted device is an auditory prosthesis such as a cochlear implant, the implanted device can lack its own sound input device (e.g., a microphone). It is sometimes utilitarian to remove the external component. For example, it is common for a recipient of an auditory prosthesis to remove an external portion of the prosthesis while sleeping. Doing so can result in loss of function of the implanted portion of the prosthesis, which can make it impossible for recipient to hear ambient sound. This can be less than utilitarian and can result in the recipient being unable to hear while sleeping. Loss of function would also prevent the implanted portion from responding to signals representative of streamed content (e.g., music streamed from a phone) or providing other functionality, such as providing tinnitus suppression noise.

The external component that provides power and/or data can be worn by the recipient, as detailed above. While a wearable external device is worn by a recipient, the external device is typically in very close proximity and tightly aligned with an implanted component. The wearable external device can be configured to operate in these conditions. Conversely, in some instances, an unworn device can generally be further away and less tightly aligned with the implanted component. This can create difficulties where the implanted device depends on an external device for power and data (e.g., where the implanted device lacks its own battery and microphone), and the external device can need to continuously and consistently provide power and data in order to allow for continuous and consistent functionality of the implanted device.

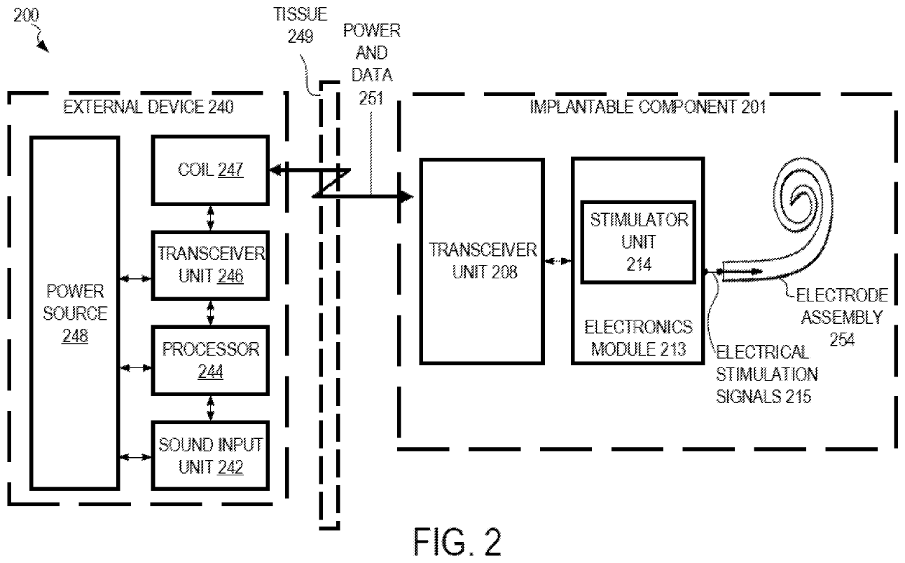

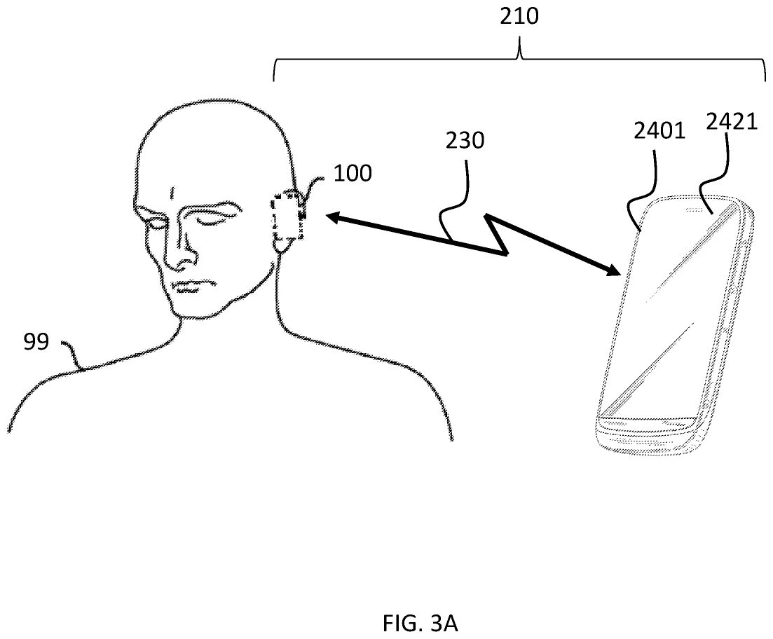

FIG. 2 is a functional block diagram of a cochlear implant system 200 to which the teaching herein can be applicable. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Publication Number 2009/0216296 to Meskens. In this manner, the data signal is modulated with the power signal, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power, because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device. In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to the stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

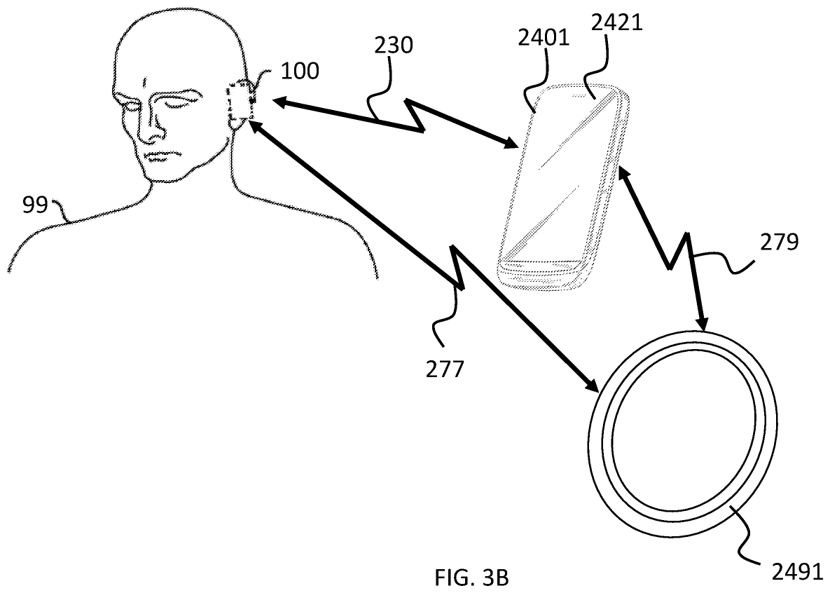

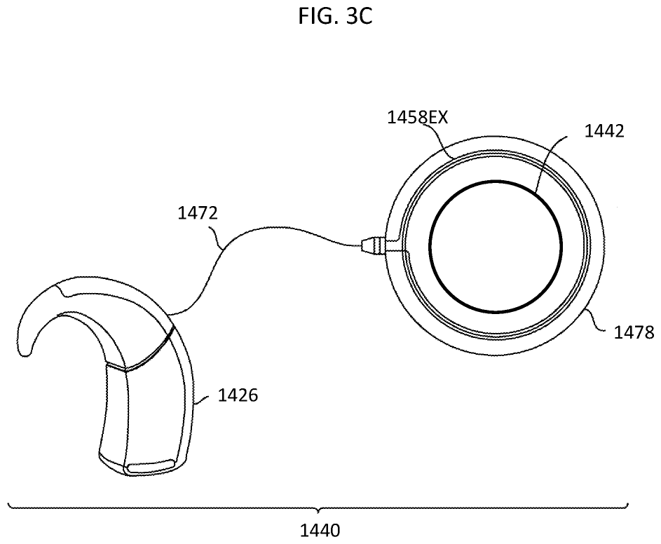

FIG. 3A depicts an exemplary system 210 according to an exemplary embodiment, including hearing prosthesis 100, which, in an exemplary embodiment, corresponds to cochlear implant 100 detailed above, and a portable body carried device (e.g., a portable handheld device as seen in FIG. 2A, a watch, a pocket device, etc.) 2401 in the form of a mobile computer having a display 2421. The system includes a wireless link 230 between the portable handheld device 2401 and the hearing prosthesis 100. In an embodiment, the prosthesis 100 is an implant implanted in recipient 99 (represented functionally by the dashed lines of box 100 in FIG. 3A).

In an exemplary embodiment, the system 210 is configured such that the hearing prosthesis 100 and the portable handheld device 2401 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 2401 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 2401 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 100. Some of these embodiments will be described below).)

In an exemplary embodiment, the portable handheld device 2401 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 2401 is also encompassed by such disclosure, unless otherwise specified or otherwise incompatible with the pertinent technology (and vice versa).

It is noted that in some embodiments, the system 210 is configured such that cochlear implant 100 and the portable device 2401 have a relationship. By way of example only and not by way of limitation, in an exemplary embodiment, the relationship is the ability of the device 2401 to serve as a remote microphone for the prosthesis 100 via the wireless link 230. Thus, device 2401 can be a remote mic. That said, in an alternate embodiment, the device 2401 is a stand-alone recording/sound capture device.

It is noted that in at least some exemplary embodiments, the device 2401 corresponds to an Apple Watch™ Series 1 or Series 2, as is available in the United States of America for commercial purchase as of Jan. 10, 2021. In an exemplary embodiment, the device 2401 corresponds to a Samsung Galaxy Gear™ Gear 2, as is available in the United States of America for commercial purchase as of Jan. 10, 2021. The device is programmed and configured to communicate with the prosthesis and/or to function to enable the teachings detailed herein.

In an exemplary embodiment, a telecommunication infrastructure can be in communication with the hearing prosthesis 100 and/or the device 2401. By way of example only and not by way of limitation, a telecoil 2491 or some other communication system (Bluetooth, etc.) is used to communicate with the prosthesis and/or the remote device. FIG. 3B depicts an exemplary quasi-functional schematic depicting communication between an external communication system 2491 (e.g., a telecoil, or Bluetooth transceiver), and the hearing prosthesis 100 and/or the handheld device 2401 by way of links 277 and 279, respectively (note that FIG. 3B depicts two-way communication between the hearing prosthesis 100 and the external audio source 2491, and between the handheld device and the external audio source 2491—in alternate embodiments, the communication is only one way (e.g., from the external audio source 2491 to the respective device)). It is noted that unless otherwise noted, the embodiment of FIG. 3B is applicable to any body worn medical device/implanted device disclosed herein in some embodiments.

FIG. 3C depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

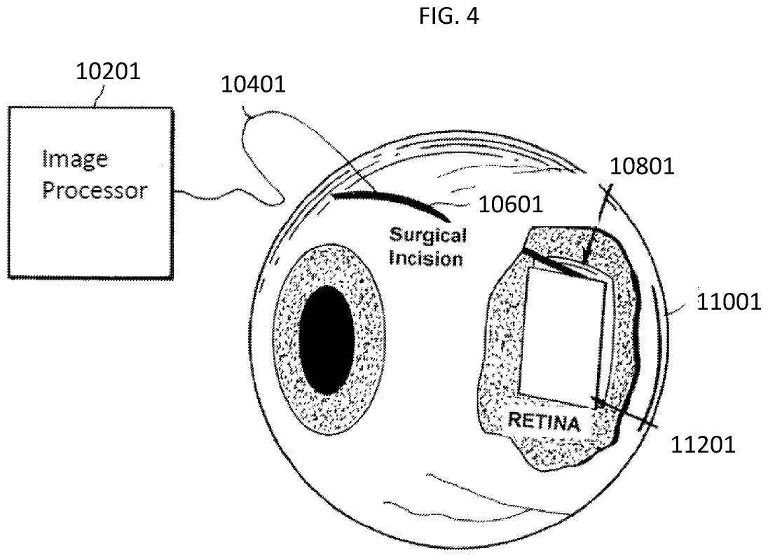

FIG. 4 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

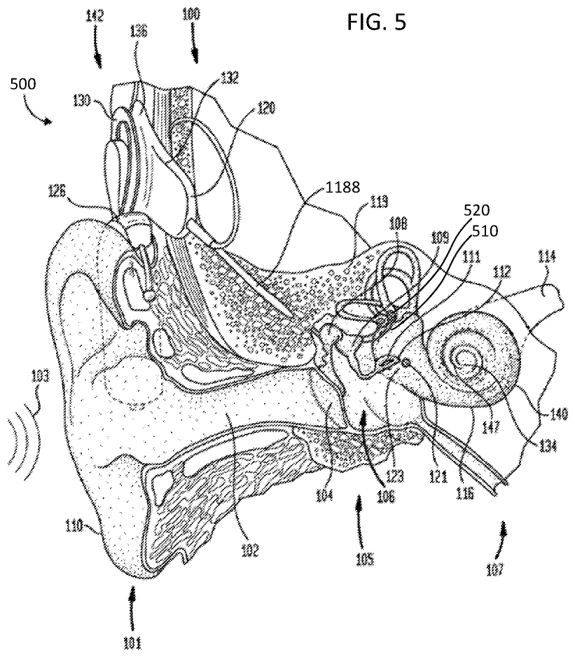

FIG. 5 depicts an exemplary vestibular implant 500 according to one example. Some specific features are described utilizing the above-noted cochlear implant of FIG. 1 in the context of a vestibular implant. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 500 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith.

The implantable component includes a receiver stimulator in a manner concomitant with the above cochlear implant. Here, vestibular stimulator comprises a main implantable component 120 and an elongate electrode assembly 1188 (where the elongate electrode assembly 1188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 1188.

It is briefly noted that while the embodiment shown in FIG. 5 represents a partially implantable vestibular implant, embodiments can include a totally implantable vestibular implant, such as, where, for example, the motion sensors are located in the implantable portion, in a manner analogous to a cochlear implant.

Elongate electrode assembly 1188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 1188 includes a plurality of leads 510 that branch out away from the main body of the electrode assembly 118 to electrodes 520. Electrodes 520 can be placed at the base of the semicircular ducts as shown in FIG. 5. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

At least some exemplary embodiments according to the teachings detailed herein utilize advanced learning processing techniques, which are able to be trained to detect higher order, and non-linear, statistical properties of data. An exemplary analytical technique is the so called deep neural network (DNN). At least some exemplary embodiments utilize a DNN (or any other advanced learning analytical technique) to analyze a person's demographic data and other data relating to sensory characteristics (e.g., hearing loss) and other characteristics (e.g., a person's willpower or beliefs about sensory aids)—more on all this below. At least some exemplary embodiments entail training data analysis algorithms/developing models to detect subtle and/or not-so-subtle changes, and provide an estimate of future statuses and conditions, etc., and specific information thereabout. That is, some exemplary methods utilize learning algorithms such as DNNs or any other algorithm that can have utilitarian value where that would otherwise enable the teachings detailed herein to analyze a person's data to predict certain characteristics/actions/occurrences, etc.

A “neural network” is a specific type of machine learning system. Any disclosure herein of the species “neural network” constitutes a disclosure of the genus of a “machine learning system.” Moreover, any disclosure herein of the species “machine learning” constitutes a disclosure of the genus of “artificial intelligence.” While embodiments herein focus on the species of a neural network, it is noted that other embodiments can utilize other species of machine learning systems accordingly, or the broader genuses noted, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Some of the specifics of the DNN utilized in some embodiments will be described below, including some exemplary processes to train such DNN. First, however, some of the exemplary methods of utilizing such a DNN (or any other system that can have utilitarian value) will be described.

It is noted that in at least some exemplary embodiments, the DNN or the product from machine learning, etc., or the results thereof, etc., is utilized to achieve a given functionality as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product or results from machine learning, etc., that when used, results in that functionality, unless otherwise noted or unless the art does not enable such.

With the above in mind, there are many types of medical conditions that can be treated in a variety of manners, including the utilization of medical devices. Some treatments are more invasive or otherwise “traumatic” than others, and there can be hesitancy by people to adopting some of the more aggressive or invasive treatments. In this regard, by way of example only and not by way of limitation, the medical conditions associated with sensory loss, such as for example, hearing loss will be focused on below. It is noted that the teachings detailed thereabout are representative and constitute a disclosure of the utilization of those teachings and other conditions, such as, for example, vision loss or balance loss or tactile loss or taste or smell loss, etc. and also, the medical conditions need not be associated with sensory loss in some embodiments. Instead, it can be some form of medical condition unrelated to the senses, and can also be associated with treatments that do not directly utilize medical devices, at least not prostatic or medical devices that are used by the person suffering the medical condition, as distinguished from a clinician or a medical professional treating the medical condition. Accordingly, the disclosure herein constitutes a representative disclosure, and such disclosures also correspond to another disclosure of utilizing these representative teachings with these other scenarios. Other scenarios can include atrial fibrillation, high blood pressure, myopia, dementia, Alzheimer's, sleep apnea, allergies, epilepsy, fatigue, osteoporosis, diabetes, obesity, etc.

Still, returning back to the more invasive and traumatic treatments for sensory loss, there are many barriers in the process of a candidate or potential candidate to receive a sensory implant, such as a cochlear implant to treat hearing loss, or a retinal implant to treat vision loss. This can be attributed to lack of awareness or uncertainty of, for example, cochlear implantation and its efficacy in treating severe to profound hearing loss, and the potential risks involved during surgery. These barriers can affect the candidate and the potential candidate, the candidate's loved ones, and the clinical professional providing care, for example. The occurrence of the barriers can be in the hearing aid audiology hearing treatment efforts and the cochlear implantation treatment efforts. Put another way, the utilization of a cochlear implant requires a surgery that results in an electronic device being implanted in the head of a person on the other side of the skull from the brain. Moreover, the electrodes are placed within an inch or two of the brain, and otherwise provide stimulation to the nervous system of the human. Corollary to this is that there are risks of infection and there are risks of further hearing loss, such as if, for example, the candidate or potential candidate has residual hearing. Moreover, with present technology, the utilization of a cochlear implant could foreclose other treatments of restoring hearing, such as, for example, regrowth of the cilia in the cochlea. This could be something that prevents a potential candidate from receiving the cochlear implant because he or she believes that there will be some treatment developed in the future that will restore his or her hearing without surgery and/or will restore his or her hearing in a manner superior to that which results from a cochlear implant. This could be a strongly held belief, yet for somebody who is 70 or 80 years old, it is a very unlikely scenario with respect to the actuarial data associated with that person.

To overcome the barriers, an application containing a suite of technologies provides medical treatment counseling, such as sensory aid or hearing device utilization counseling, including cochlear implantation counselling, information personalized to the potential candidate and candidate. Clinical professionals (clinician) will use this application during the counselling process when providing counsel to perspective recipients of a cochlear implant for example or of an active transcutaneous bone conduction device or a middle ear implants or a retinal prosthesis all by way of example vis-a-vis sensory supplement devices. The process includes obtaining candidate details (the clinician could enter this, or the candidate could enter this, for example, into a computer or a website, or this information can be retrieved from a database where the data was previously obtained, etc.) such as, for example demographics, medical history, hearing history and the candidate's responses to questions. The suite of technologies comprising the application processes the inputted data using models and algorithms (herein, any disclosure of the former corresponds to a disclosure of the latter, and vis-a-versa, in the interests of textual economy) and outputs personalized counselling information. In language relevant to the clinician, the candidate and the candidate's loved ones, the personalized counselling information can convey: (i) how well the candidate may hear in comparison to their pre-treatment state; (ii) how well does a cohort of similar sensory aid recipients, such as cochlear implant recipients, perform; (iii) how will the candidate's hearing improve overtime post-aid use/post-implantation (iv) what functional activities the candidate may be able to engage in; (v) what will be the impact and risks if the candidate decides to delay treatment; and/or (vi) what should the candidate expect and what activities they must engage in post-beginning of aid use/post implantation.

Embodiments thus can include devices, systems, and methods that can provide enhancement of preoperative candidate (or potential candidate) counselling conducted by a clinician professional with personalized predictions of outcomes (variants of clinical measures and functional activities for example) and tailored information and messaging in the language of the clinician and candidate conducted by models/algorithms within an application. As noted above, embodiments can utilize artificial intelligence to implement at least some of these teachings.

In an exemplary embodiment, some exemplary functional activities can be the detection and/or recognition of environmental sounds such as for example, alarms, alerts, horns, traffic, etc., the ability to participate in a conversation with another person in a quiet environment, another person in a noisy environment and/or a group conversation in a quiet environment. In an exemplary embodiment, some exemplary functional activities can be the ability to participate in a telephone conversation, listen to the television, localize the source of different sounds or differentiate sounds (directionally and/or in three dimensions) and/or appreciate music. All of these are qualified with respect to the understanding of the person of ordinary skill in the art which could be a trained audiologist with 10 years of experience and/or a trained hearing specialist, including and otherwise an eye, ear nose and throat doctor, or a hearing doctor, licensed in the United States of America and/or any of the states of the United States, including, for example, any of the states listed herein, the European Union, the United Kingdom, the Republic of France, the People's Republic of China, the Republic of Germany, Australia, Japan, and/or New Zealand. For example, the functional activity of appreciating music is a qualitative feature that would be understood by the just-noted persons of skill. Conversely, the ability to distinguish three or four or five different notes within an octave lasting less than a second for example 90% of the time would be a qualitative measure. Any quantitative and/or qualitative measure of the functional activity and/or any functional activity that can be utilized to gauge the level of functional activities in which the candidate will engage can be utilized in at least some exemplary embodiments, providing that the art enable such and providing that such has utilitarian value. In an embodiment, functional activities can include any of the ability to:
a. Appreciate music
b. Listen to the television
c. Participate in one-on-one conversations
d. Engage in conversation in group settings
e. Use the telephone for conversations
f. Recognize environmental sound, alerts and alarms
g. Listen to streamed audio from the hearing device with Bluetooth connectivity
h. Participate in employment
i. Participate in charities

In an exemplary embodiment, any of the functionalities detailed herein can correspond to an ability to engage in that functionality corresponding to, at a minimum, a performance at which would exist for a 20, 25, 30, 35, 40, 45, 50, 55, 60, 65, 70, 75, or 80th percentile, or any value or range of values therebetween in 1% increments human factors engineering human of 5, 10, 15, 20, 25, 30, 35, 40, 45, 50, 55, 60, 65, 70, 75, or 80 years of age or older, or any value or range of values therebetween in 1 year increments who is a natural born citizen of any of the jurisdictions detailed herein and/or a natural born citizen in the jurisdiction in which the candidate has lived his or her sensory and communicative formative years. The age could be the same as the candidate or could be different, and can be within 5, 10, 15, 20, 25, 30, 35, 40, 45, 50, 55, 60, 65, 70, 75, or 80 years or more of the candidate. The human factors engineering values can be broken out for gender, and the control values could be limited to those of the same gender as the candidate. Moreover, the human factors engineering values can be reduced to specificity, such as for example hearing curves for different people or vision curves for different people. These performative measures are as of Dec. 21, 2022, if such data is available, and/or for the date in which the methods herein are executed and/or the device and/or system is used, as is the case for all comparative measures herein. Thus, to appreciate music for example, the appreciation will correspond to, for example, the appreciation that would result from at least the 30^{th} percentile human factors engineering human within five years of a person who has hearing difficulties who has received a cochlear implant or who is a candidate for receiving a cochlear implant. The hearing appreciation can be reduced to quantitative measures which quantitative measures would correspond to that which would exist for the aforementioned 30^{th} percentile human factors engineering human. This aforementioned person could have the hearing of a 30^{th} percentile human factors engineering human. Qualitatively, this could be reduced to a binary concept, such as was listening to your favorite music enjoyable. A person who is hard of hearing may not find listening to his or her favorite music enjoyable if he or she cannot hear the music. And favorite music could be reduced to, for example, a song or music that the person likes, and this can be a narrow subset of music. For example, rendition of some form of classical music that the person likes (the recording is what he/she likes) played by a high-end stereo at 70 dB, a person who is a Barry White fan (or not even a fan) will certainly know whether Love's Theme as played from a high-end stereo system at 40, 45, 50, 55, 60, 65 or 70 dB or any value or range of values therebetween in 1 dB increments is enjoyable, and even non-Barry White fans, at least above a certain age. Olivia Newton John's “Magic” and “Call Me” and “Another Brick In the Wall” and “Ghostbusters” are additional examples. The same could be the case for heavy metal, one's favorite elevator music, etc. (The aforementioned music was charted for significant amounts of time in the past.)

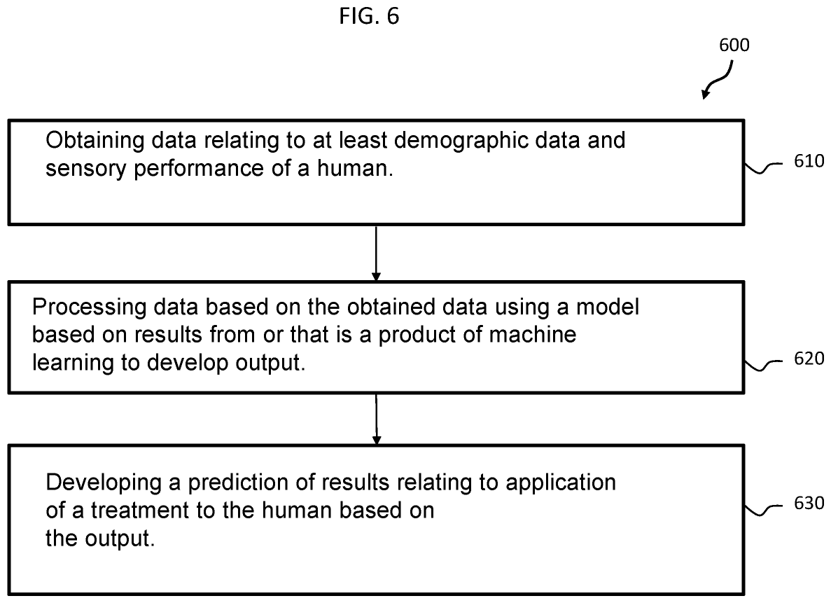

As an initial concept, briefly, FIG. 6 depicts an exemplary flowchart for an exemplary method, method 600, of utilizing a model based on results from and/or that is a product of artificial intelligence, such as, for example, machine learning, such as a DNN, according to an exemplary embodiment. Method 300 includes method action 310, which includes obtaining data relating to attributes of a human. In an exemplary embodiment, such as that focused on a hearing assistance device, the obtained data relating to attributes of the human is one or more of any of the following:

| | |
|---|---|
| Age of the human | Whether language spoken by the |
| Audiogram of the human | human was learned prior to or after |
| Duration of hearing loss of the | the onset of hearing loss |
| human | Pre-sensory aid (e.g., preoperative) |
| Duration of using some form of | speech test results of the human |
| hearing aid of the human | Cognitive assessments of the human |
| Rate of hearing loss of the human | Mental health status questionnaire |
| Cause of hearing loss of the human | results of the human |
| Anticipated sensory aid usage by | Medical health status questionnaire |
| the human | results of the human |
| Comorbidities of the human | Sensory (e.g., hearing) quality of |
| | life questionnaire of the human |

Additional data relating to the human can be obtained. Any data that can enable the teachings detailed herein and/or otherwise provide utilitarian value can be used providing that the art enables such.

As will be understood, the human that is the subject of method 600 can be a human who is a candidate for a sensory aid, such as a cochlear implant or retinal implant.

Method action 610 can be executed by asking the human questions, having the human answer the questions, and/or having the human complete a written questionnaire. Method action 610 can be executed telephonically or over the Internet. Method action 610 can be executed by providing the person with an electronic computing device or otherwise an electronic input device, such as electronic notepads that are commonly available at doctor's offices. A smart phone application can be utilized to obtain the data. Any device, system, and/or method that can enable the action of obtaining data relating to the human can be utilized in at least some exemplary embodiments. Thus, in an alternative embodiment, the data can be obtained from an entity that obtained and/or analyzed original base data. That is, in an exemplary embodiment, to execute method action 610, the actor need not necessarily be the person who directly obtains the raw data/base data.

It is also noted that in at least some exemplary embodiments, method action 610 can be executed such that the person who is the subject of the method action is at a remote location from the entity obtaining the data/the entity executing method action 610. By way of example only and not by way of limitation, in an exemplary embodiment, the person can be speaking to a telephone, as noted above, and the telephone can transmit the person's speech to a remote facility, anywhere in the world in some embodiments, where the person's speech is received by the entity executing the method. Alternatively, the person can enter the information into an app on a smart phone, or fill out a web page, and the data can be transmitted to the remote location. Any device, system, and/or method that can enable the information to reach the person or entity executing method 600 can be utilized, providing that the art enables such and such provides utilitarian value.

And also, the data need not necessarily be obtained directly from the human to execute method action 610. In an exemplary embodiment, method action 610 can be executed by accessing a database where the data is stored. Also, method action 610 can be executed by obtaining data that has been manipulated or otherwise organized or “weighted” or filtered or subject to some other data processing regime.

As seen from FIG. 6, in some embodiments, method action 610 includes obtaining data that is tailored to specific subsets of the human, such as data relating to at least demographic data and sensory performance of the human.

In an embodiment, the demographic data includes age, data relating to hearing loss and speech attributes (it can include others), and where the method is used to develop/obtain a prediction of results relating to application of a sensory supplement device to the human, the sensory supplement device can be a cochlear implant, an active transcutaneous bone conduction device, a middle ear implant, all by way of example.

Method 600 further includes method action 620, which includes analyzing data based on the obtained data using a model based on results from or that is a product of machine learning to develop output. In an exemplary embodiment, this can correspond to analyzing the raw data that is obtained in method action 610. Further, this action of method action 620 can correspond to analyzing modified data or filtered data or transformed data (hence analyzing data based on the obtained data—this can be the data or data that has been transformed). In an exemplary embodiment, the product is a chip that is fabricated based on the results of machine learning. In an exemplary embodiment, the product/result is a neural network, such as a deep neural network (DNN). The product can be based on or be from a neural network. In an exemplary embodiment, the product/results is code. In an exemplary embodiment, the product/results is a logic circuit that is fabricated based on the results of machine learning. The product/results can be an ASIC (e.g., an artificial intelligence ASIC). The product/results can be implemented directly on a silicon structure or the like. Any device, system, and/or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems, and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network. The teachings detailed herein include utilizing machine learning methodologies and the like to establish sensory prosthetic devices or supplemental components utilized with sensory prostatic devices (e.g., a smart phone), to replace or otherwise augment the processing functions, etc. (e.g., sound or light processing, etc.) of a given sensory prostheses.

As noted above, method action 620 entails analyzing the data utilizing a product of or a model that results from machine learning, such as the results of the utilization of a DNN, a machine learning algorithm or system, or any artificial intelligence system that can be utilized to enable the teachings detailed herein. This as contrasted from, for example, analyzing the data utilizing general code or utilizing code that not from a machine learning algorithm or utilizing a non-AI based/resulting chip, etc.

Again, in an exemplary embodiment, the machine learning can be a DNN, and the product/results can correspond to a trained DNN and/or can be a product based on or from the DNN (more on this below).

In an embodiment, method action 620 is executed using a code of and/or from a machine learning algorithm, an AI algorithm, etc., to develop the output. In an exemplary embodiment, the machine learning algorithm can be a DNN, and the code can correspond to a trained DNN and/or can be a code from the DNN.

FIG. 6 further includes method action 630, which includes developing a prediction of results relating to application of a treatment to the human based on the output. In an exemplary embodiment the treatment is an application of a sensory supplement device, such as a hearing aid or a cochlear implant, and thus method action 630 includes developing a prediction of results relating to application of a sensory supplement device to the human based on the output.

The sensory supplement device can be a retinal implant for example. The sensory supplement device can be a device that amplifies or otherwise increases a volume of sound, whether that is something that is located in the ear canal, or exterior to the ear canal. The sensory supplement device could be a bone conduction device, whether percutaneous or transcutaneous (active or passive). In an embodiment, the sensory supplement device could be a cochlear implant or a middle ear implant. Accordingly, it can be seen that in some embodiments, the sensory supplement device is a hearing supplement device. Any device that can aid a human in hearing or otherwise understanding speech directed to the human can be a hearing supplement device. In an exemplary embodiment, the sensory supplement device is a hearing prosthesis, such as a hearing aid (such as a device one can obtain based on a prescription from an audiologist for example) or an active transcutaneous bone conduction device or a middle-ear implant or a cochlear implant, or a consumer electronics hearing assist device (which is a non-prescription device-hearing aids can be non-prescription devices-such as a headset that cancels some noise and/or increases a volume of output from a speaker based on input, such as captured sound captured from a microphone).

It is briefly noted that in some embodiments, the output of method action 620 can be the prediction. That is, by way of example, the device that is utilized to analyze the data can also produce the prediction, whereas in other embodiments, a separate device or subsystem is utilized to develop the prediction based on the output. This could be a limitation of the device that is utilized to perform the analysis, or this could be a function of the fact that the output of the device is not in a prediction quality per se. For example, if the output is that there is a 35% to 45% chance that a person will be able to speak on a telephone without a text device with a person who also speaks the same language in the above-noted scenario, the prediction would be that it is more likely than not that the person will not be able to do so. This is a simple example of how output is utilized to develop a prediction. Still further by example, the device that analyzes the data could output the prediction.

Thus, in an embodiment, there is a method, including the action of executing method action 610, and executing method action 620, wherein at least one of: (i) the output is a prediction of results relating to application of a sensory supplement device to the human; or (ii) the method further includes developing a prediction of results relating to application of a sensory supplement device to the human based on the output.

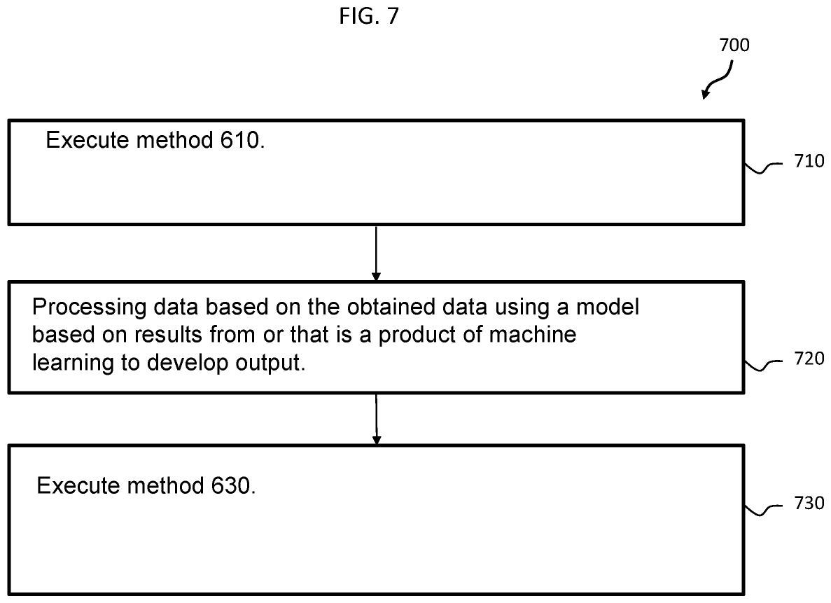

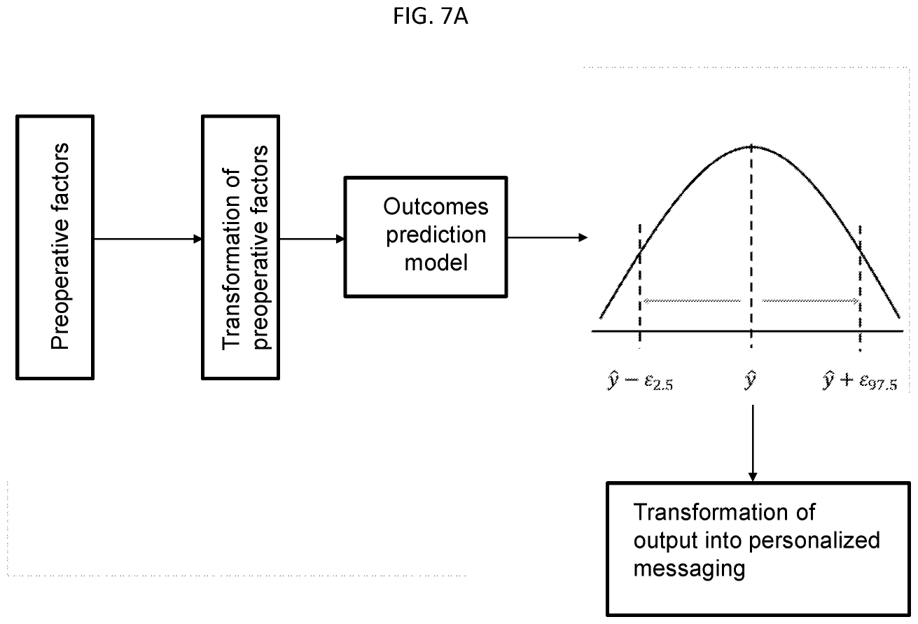

FIG. 7 presents an expanded method, method 700. Method 700 includes method action 710, which includes executing method action 610. Method 700 also includes method action 720, which includes analyzing data based on the obtained data obtained in method action 710 using a statistical model, a probabilistic model and/or a model based on results from or that is a product of machine learning to develop output. Method 700 also includes method action 730, which includes executing method 630.

In an embodiment, an outcomes prediction model is used to execute at least some of the actions of method 600 and method 700 (and some other methods, as the art enables such in a manner having utilitarian value). The aforementioned model can be the outcomes prediction model. In an embodiment, the developed predictions are predictions of clinical outcomes measures, and in other embodiments, the developed predictions are predictions of layman performance/functionality (more on this in a moment), and in some embodiments, there is a combination of these, and other types of measures. This can be achieved, as can be seen, by obtaining (having the candidate for a hearing prosthesis/or a clinician inputting) preoperative factors, details or information about a human who is a candidate for a sensory aid, into a statistical, probabilistic or machine learning model. The model then predicts, for example, a magnitude and/or range (i.e., prediction interval) of the outcomes measure.

In an embodiment, the prediction models detailed herein are results of machine learning/a product of machine learning (either directly or indirectly/based thereon), or a DNN, etc., that has the functionality thereof, and executes the functionality based on input thereto in view of its training. The prediction model can be a computer chip (as opposed to a processor) or an electronic circuit. The prediction model can be electronics that have the function thereof. Consistent with the well-known phenomenon of machine learning, it may not be that the prediction model can be specifically understood. It may not be known exactly how the prediction model works, consistent with how artificial intelligence works in general and machine learning works specifically. The prediction model can be the results of the machine learning as noted above. This can be the machine learning as frozen in time (when the learning was halted to establish the product). In an exemplary embodiment, an algorithm can include and/or the models herein can be based on a linear model, such as y=β₀+Σβᵢxᵢ+ε. Here, y is the outcome being predicted, β are coefficients and x are inputs.

The clinical outcomes measures can be in the form of speech tests such as by way of example CNC word test, CVC word test, Freiburg Monosyllabic word test, Azbio sentence test, etc., as those tests exist and as adopted by the industry in the United States, nationally or by the pertinent regulatory authority in any one of the individual states, such as New York, California, Texas, New Jersey, Florida or Utah, the European Union, the United Kingdom, the Republic of France, the Federal Republic of Germany, and/or the Republic of China, on Dec. 21, 2022. The magnitude of the speech tests or the change in the speech tests between the preoperative and postoperative states (or prior to beginning treatment (e.g., use of the sensory aid, preoperative state) and post beginning of treatment (e.g., post beginning of use of the sensory aid, post operative state) may be predicted using the model(s). The time points of the post beginning of treatment states may include switch-on, 3 months, 6 months, 1 year, 2 years, etc.

Conversely, the more generalized measures can be a qualitative and/or quantitative evaluation of how well a candidate will perform on a given task. By way of example only and not by way of limitation, will a candidate be able to use a telephone without text conversion with a fluent English speaker for example or a fluent Mandarin speaker or a fluent French or German speaker or Japanese speaker from the same region of the country as the candidate of the same age or within five or 10 or 15 or 20 years of the candidate speaking in proper English/Mandarin/French/German/Japanese, etc., or more subjectively, his or her grandchildren by way of example. More on this below.

Briefly, in the interests of textual economy, any disclosure herein of a treatment corresponds to a disclosure of a general treatment of an ailment and/or a specific treatment, such as sight loss, hearing loss, high blood pressure, dizziness, sleep apnea, lack of concentration, atrial fibrillation, etc., and vice versa (any of these), providing that the art enables such and providing that such as utilitarian value. Any disclosure herein of a treatment corresponds to a disclosure of the use of a medical device, such as a sensory prosthesis, such as a cochlear implant, a conventional hearing aid, a bone conduction device (active transcutaneous, passive transcutaneous and/or percutaneous), a middle ear implant, a retinal prosthesis, or a non-medical device, such as a voice to text device, or a noise cancellation system, and vice versa (any of these), providing that the art enables such and providing that such as utilitarian value. Any disclosure herein of preoperative and post operative corresponds to a disclosure of pretreatment and post beginning of treatment and/or pre use of a sensory aid (medical device or nonmedical device any of those disclosed herein) and post beginning of use of the sensory aid vice versa (any of these), providing that the art enables such and providing that such as utilitarian value. The point is that herein, these phrases may be utilized with respect to a specific embodiment, such as a cochlear implant, for purposes of illustration and ease of explanation. To avoid the repetitiveness of explaining that one feature is associated with another feature of another embodiment, this standardized disclosure is presented and any deviation from such will be pointed out herein.

In an embodiment, the statistical, probabilistic and/or machine learning models, etc., are developed by analyzing utilitarian amounts of historically collected treatment data (e.g., preoperative and postoperative data of cochlear implant patients). In an embodiment, depending on the data collected and/or the nuances of the population associated with the data that is collected (if the population is statistically similar in a given category, less data will be needed than if the population has a high number of people who are statistically different in a given category) the amounts of collected data can correspond to data for less than greater than or equal to 70, 80, 90, 100, 125, 150, 175, 200, 250, 300, 350, 400, 500, 600, 700, 800, 900, 1000, 1250, 1500, 1750, 2000, 3000, 4000, 5000, 6000, 7000, 8000, 9000, or 10000 people, or any value or range of values therebetween in 1 increment (e.g., 73, 555, 321 to 4,444 people, etc.).

The data analyzing algorithms to develop the model(s) can include by way of example:
(i) curation of the raw dataset/raw historical data to remove and/or adjust data of patients whose records are sparely populated or are of low quality;
(ii) imputation of the dataset/raw historical data to fill in missing data using patterns and relationships within the dataset;
(iii) transformation of information such as cause of hearing loss or sight loss or whatever ailment is at issue and/or outputs of questions of questionnaires into numerical forms;
(iv) transformation of preoperative factors in principle components (using principal component analysis) or independent components (independent component analysis);
(v) identification of preoperative factors and/or transformed factors that correlate and/or causally relate to the outcomes metrics; and/or
(vi) machine learning training (or AI training), which can include calculation of coefficients, parameters, thresholds and/or weights of models by minimising a cost function such as minimising the sum of squared error.

FIG. 7A presents a process flow diagram for a programmatic flow for the outcomes prediction model(s). Pretreatment/preoperative factors are inputted using for example, a user interface (as detailed herein for example) of an application designed to collect/obtain data from the candidate (directly or through another person, such as a clinician). Then, the factors are transformed according to the methods identified during model development. The transformed factors are inputted into the model. The model produces a central estimate of the outcomes measure (ŷ), a corresponding prediction interval ({ŷ-ε₂₅, ŷ−ε_{95.7}}) and a probability density function (PDF) which describes the probability densities of different magnitudes occurring. The output of the model is transformed into natural language that can be used during the counselling conversation. The natural language messaging will be described below, but briefly, the natural language messaging can be the prediction.

Returning back to methods 600 and 700, in an embodiment where the sensory performance is hearing, the prediction can be a magnitude of a result of a speech test and/or a change in result of a speech test. That is, for example, in an embodiment, the prediction can be that the person or candidate will improve on a speech test by a certain amount after receiving a cochlear implant for example, and utilizing the cochlear implant for a certain period of time, such as three months or six months etc. Alternatively, in an embodiment, the prediction can be that a person will receive a score of between X and Y on a certain standardized test for example.

In an embodiment, the prediction of methods 600 and 700 can be a prediction regarding functional activities in which the human can engage. The above-noted example regarding the use of a telephone without text conversion is an example of a functional activity. The ability to hear a telephone ring, or an alarm clock, or the ability to hear meaningfully in a room where two, three, four or more people are present who all speak at different times is a functional activity. Hearing a television or a radio and understanding the speech without captioning is a functional activity. Having a one-on-one conversation with a coworker is a functional activity.

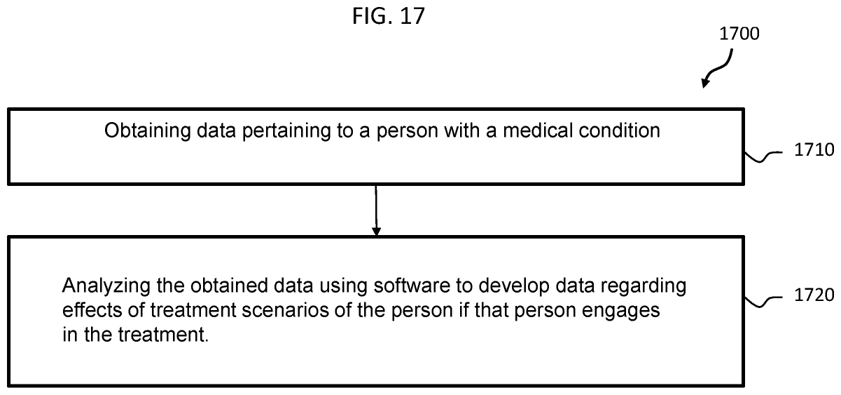

FIG. 17 shows an exemplary algorithm for a method, method 1700, according to an exemplary embodiment. Method 1700 includes method action 1710, which entails obtaining data pertaining to a person with a medical condition. This can be performed according to any of the data obtaining actions or techniques detailed herein or any others providing that the art enables such. Method 1700 further includes method action 1720, which includes the action of analyzing the obtained data using a computer chip or a logic circuit or electronics or software, to develop data regarding effects of treatment scenarios of the person if that person engages in the treatment. In an exemplary embodiment, the treatment scenarios can include a scenario where the person is given a sensory aid, such as a sensory prosthesis, such as a cochlear implant or a retinal implant, or any of the other sensory devices or hearing devices detailed herein. In an embodiment, the aforementioned a computer chip or a logic circuit or electronics or software, is based on a statistically significant population of persons who were afflicted with the medical condition and who also engaged in the treatment.

Thus, in an embodiment, the treatment of method action 1710 is the use of a given sensory aid and the a computer chip or a logic circuit or electronics or software is based on a statistically significant population of sensory impaired persons who obtained the given sensory aid. Consistent with the teachings herein, in an embodiment, the a computer chip or a logic circuit or electronics or software is code of and/or from an AI algorithm, such as a machine learning algorithm, and the AI algorithm (e.g., the machine learning algorithm) is part of a trained system trained based on a statistically significant population of sensory impaired persons who obtained the given sensory aid. In an embodiment, the person is a hearing impaired person, the sensory aid is a hearing device (e.g., a hearing aid, a cochlear implant, or a commercially available hearing assist device) and the sensory impaired persons are hearing impaired persons.

In an embodiment, the scenarios are based on performance of one or more United States medical community industry accepted sensory tests, and/or sensory tests that are accepted by the pertinent regulatory authority in any one or more of the states of Texas, New Jersey, New York, Utah, California and/or Florida as of Dec. 21, 2022 and/or adopted the pertinent regulatory authority in any one or more of the European Union, the United Kingdom, the Republic of France, the Federal Republic of Germany, and/or the Republic of China on that date.

In an embodiment, the scenarios include qualitative values pertaining to at least two functional outcomes, and/or 1, 2, 3, 4, 5, 6, 7, 8, 9, or 10 or more, or any value or range of values therebetween in 1 increment) relating to an improved sensory experience resulting from use of the sensory aid. In an embodiment, the scenarios include performance expectations using the sensory aid after dedicated practice with the sensory aid by the person. This is contrasted to, for example, the mere scenario where a person obtains the sensory aid and then only uses it sporadically or otherwise utilizes the sensory aid in a manner that does not “tax” or otherwise challenge the recipient of the sensory aid. By way of example only and not by way of limitation, there are recognized training regimes for people who receive cochlear implants and otherwise activities that a cochlear implant recipient is recommended to engage in so as to better enhance the efficacy of such. An exemplary embodiment can include the use of quality of life increasing training regimes, clinical measures of hearing performance boosting training regimes, etc. By way of example only and not by way of limitation, in an exemplary embodiment, a recipient of a cochlear implant can utilize sound matching drills to better familiarize himself or herself with sounds, such as sounds from horns, alarms, lawnmowers, male-female speech recognition, anger versus loud recognition, etc.

Embodiments include a system and/or a device in which some of the methods/and functions detailed herein can be implemented. It is noted that there can be a wide variety of data (input data) collection techniques and/or acquisition techniques, whether the data be utilized for the methods detailed herein with respect to predictions/forecasting, or for developing the data to train the learning algorithm's detailed herein. Such can entail the utilization of a smart phone, a personal computer, a landline phone, etc. In this regard, in an exemplary embodiment, the data can be obtained from remote locations and analyzed at a different location. It is also noted that a wide variety of data output utilization or transfer techniques can be utilized. By way of example only and not by way of limitation, in an exemplary embodiment, some of the teachings detailed herein can be utilized to remotely conduct at least portions of the method.

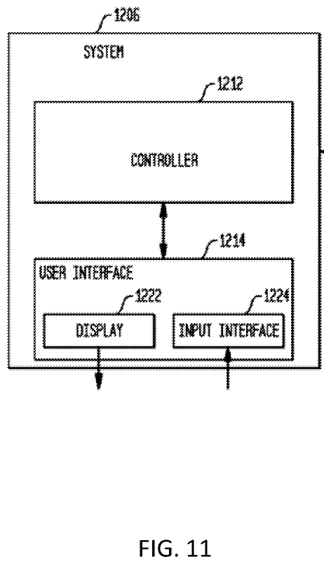

FIG. 11 presents a functional schematic of a system with which some of the teachings detailed herein and/or variations thereof can be implemented. In this regard, FIG. 11 is a schematic diagram illustrating one exemplary arrangement in which a system 1206 can be used to execute one or more or all of the method actions detailed herein. In general, in an exemplary embodiment, the system of FIG. 11 can be representative of both the system utilized to develop the model (to train the model) and the system that results from the model, or one or the other.

System 1206 will be described, at least in part, in terms of interaction with a clinician and/or a candidate for a therapy, although these terms are used as a proxy for any pertinent subject to which the system is applicable (e.g., the test subjects used to train the DNN, the subject utilized to validate the trained DNN/the professional that controls the training, the subjects and/or clinicians/professionals to which one or more of the methods are applicable, etc.). In an exemplary embodiment, system 1206 is a clinician controlled system, while in other embodiments it is a candidate controlled system, while in other embodiments, it is a remote controlled system. In an exemplary embodiment, system 1206 can correspond to a remote device and/or system, which, as detailed above, can be a portable handheld device (e.g., a smart device, such as a smart phone), and/or can be a personal computer, etc.

In an exemplary embodiment, system 1206 can be a system having additional functionality according to the method actions detailed herein. In the embodiment illustrated in FIG. 11, a remote device can be connected to system 1206 to establish a data communication link between a remote device, such as a remote computer and/or a smart phone, etc., and system 1206. System 1206 is thereafter bi-directionally coupled by a data communication link with a remote device in some embodiments. Any communications link that will enable the teachings detailed herein that will communicably couple the implant and system can be utilized in at least some embodiments.

System 1206 can comprise a system controller 1212 (a processor or chip(s) or any of the teachings herein) as well as a user interface 1214. Controller 1212 can be any type of device capable of executing instructions such as, for example, a general or special purpose computer, a handheld computer (e.g., personal digital assistant (PDA)), digital electronic circuitry, integrated circuitry, specially designed ASICs (application specific integrated circuits), firmware, software, and/or combinations thereof. As will be detailed below, in an exemplary embodiment, controller 1212 is a processor or a chip or a chipset, or circuitry specialized to execute one or more of the actions/functionalities herein. Controller 1212 can further comprise an interface for establishing the data communications link 1208 the remote device (again, which is a proxy for any device that can enable the methods herein). In embodiments in which controller 1212 comprises a computer, this interface may be, for example, internal or external to the computer. For example, in an exemplary embodiment, controller 1206 and cochlear implant may each comprise a USB, FireWire, Bluetooth, Wi-Fi, or other communications interface through which data communications link may be established. Controller 1212 can further comprise a storage device for use in storing information. This storage device can be, for example, volatile or non-volatile storage, such as, for example, random access memory, solid state storage, magnetic storage, holographic storage, etc., and can store any one or more of the data elements/information pieces detailed herein.

User interface 1214 can comprise a display 1222 and an input interface 1224 (which, in the case of a touchscreen of the portable device, can be the same). Display 1222 can be, for example, any type of display device, such as, for example, those commonly used with computer systems. In an exemplary embodiment, element 1222 corresponds to a device configured to visually display a plurality of words to the candidate and/or professional.

Input interface 1224 can be any type of interface capable of receiving information from a candidate and/or professional, such as, for example, a computer keyboard, mouse, voice-responsive software, touchscreen (e.g., integrated with display 1222), microphone (e.g. optionally coupled with voice recognition software or the like) retinal control, joystick, and any other data entry or data presentation formats now or later developed. It is noted that in an exemplary embodiment, display 1222 and input interface 1224 can be the same component, e.g., in the case of a touch screen). In an exemplary embodiment, input interface 1224 is a device configured to receive input from the candidate and/or professional indicative of a choice of one or more of the plurality of words presented by display 1222.

It is noted that in at least some exemplary embodiments, the system 1206 is configured to execute one or more or all of the method actions detailed herein, where the various sub-components of the system 1206 are utilized in their traditional manner relative to the given method actions detailed herein.

In an exemplary embodiment, the system 1206, detailed above, can execute one or more of the actions detailed herein and/or variations thereof automatically, at least those that do not require the actions of a candidate and/or professional.

While the above embodiments have been described in terms of the portable handheld device obtaining the data, either directly from the candidate and/or from the professional and performing a given analysis, as noted above, in at least some exemplary embodiments, the data can be obtained at a location remote from one or more of the people using the system. In such an exemplary embodiment, the system 1206 can thus also include the remote location (e.g., clinic).

In this vein, it is again noted that the schematic of FIG. 11 is functional. In some embodiments, a system 1206 is a self-contained device (e.g., a laptop computer, a smart phone, etc.) that is configured to execute one or more or all of the method actions detailed herein and/or variations thereof. In an alternative embodiment, system 1206 is a system having components located at various geographical locations. By way of example only and not by way of limitation, user interface 1214 can be located with the clinician or candidate (e.g., it can be the portable handheld device) and the system controller (e.g., processor) 1212 can be located remote from the professional and/or the candidate By way of example only and not by way of limitation, the system controller 1212 can communicate with the user interface 1214, and thus the portable handheld device, via the Internet and/or via cellular communication technology or the like. Indeed, in at least some embodiments, the system controller 1212 can also communicate with the user interface 1214 via the Internet and/or via cellular communication or the like. Again, in an exemplary embodiment, the user interface 1214 can be a portable communications device, such as, by way of example only and not by way of limitation, a cell phone and/or a so-called smart phone. Indeed, user interface 1214 can be utilized as part of a laptop computer or the like. Any arrangement that can enable system 1206 to be practiced and/or that can enable a system that can enable the teachings detailed herein and/or variations thereof to be practiced can be utilized in at least some embodiments.

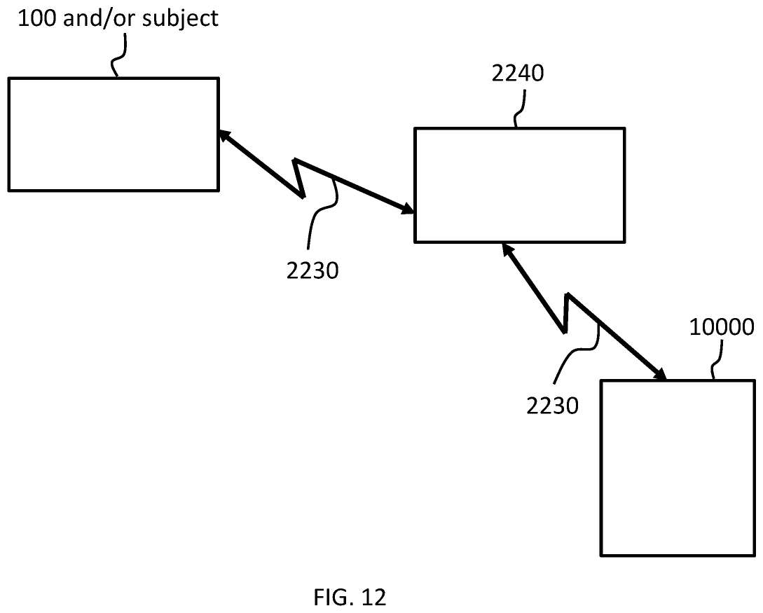

In view of the above, FIG. 12 depicts an exemplary functional schematic, where a device 2240, which will be detailed herein in this exemplary embodiment as a portable handheld device 2240 or a laptop or a smart pad or a dedicated pad or a desktop computer, etc., but is to be understood as representative of any device that can enable the teachings detailed herein (e.g., remote dedicated hearing prosthesis control unit, personal computer, smartphone, landline phone, etc.) is in communication with a geographically remote device/facility 10000 via link 2230, which can be an internet link. The geographically remote device/facility 1000 can encompass controller 1212 and/or the memory or any of the components detailed herein or that can enable the teachings herein, and the remote device 2240 can encompass the user interface 1214. The geographic remote device/facility 10000 can be the clinic or can be the medical device manufacturer's location, or a surrogate thereof. It is also noted that in the embodiment of FIG. 12, link 2230 can represent communication between the portable handheld device 2240 or laptop or smart pad or dedicated pad or desktop and/or can represent communication between the portable handheld device 2240 or a laptop or a smart pad or a dedicated pad and the subject/candidate.

Accordingly, an exemplary embodiment entails executing some or all of the method actions detailed herein where the subject/candidate, the clinician/professional and/or the portable handheld device 2240 or laptop or smart pad or dedicated pad or desktop is located remotely (e.g., geographically distant) from where at least some of the method actions detailed herein are executed.

In an exemplary embodiment, the portable handheld device 2240 or laptop or smart pad or dedicated pad or desktop is configured to execute one or more of the method actions detailed herein. In an exemplary embodiment, the device 2240 is configured to communicate with the cloud as detailed above and/or with the clinic as detailed above.

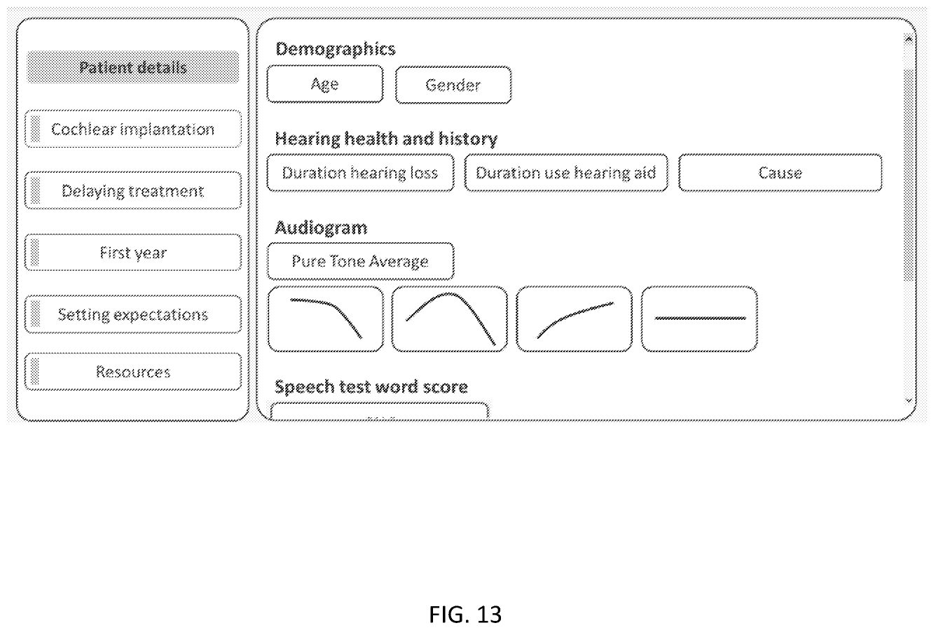

FIGS. 13-16 present example windows of a user interface according to some exemplary embodiments, where the user can be the clinician/professional and/or the candidate/subject, which windows can be displayed on a screen of a computing device or a system configured to implement the teachings detailed herein. These windows can be presented on a laptop or a tablet or even a smart phone for example, and can be presented on any of the computer components detailed herein. FIG. 13 presents the window where candidate details are entered. Details that are entered by the user about the candidate can include, by way of example only and not by way of limitation, age, gender, hearing history, hearing health, audiogram, preoperative speech test scores, and definitional details that enable algorithms and/or models to personalise of information. This can be inclusive of questionnaires such as Health Utilities Index Mark 3 (HUI 3), Speech Spatial Qualities 12 (SSQ12), Speech Spatial Qualities (SSQ49), Montreal Cognitive Assessment (Moca), etc. Any one or more or all of this can correspond to the data that is obtained in method action 610.

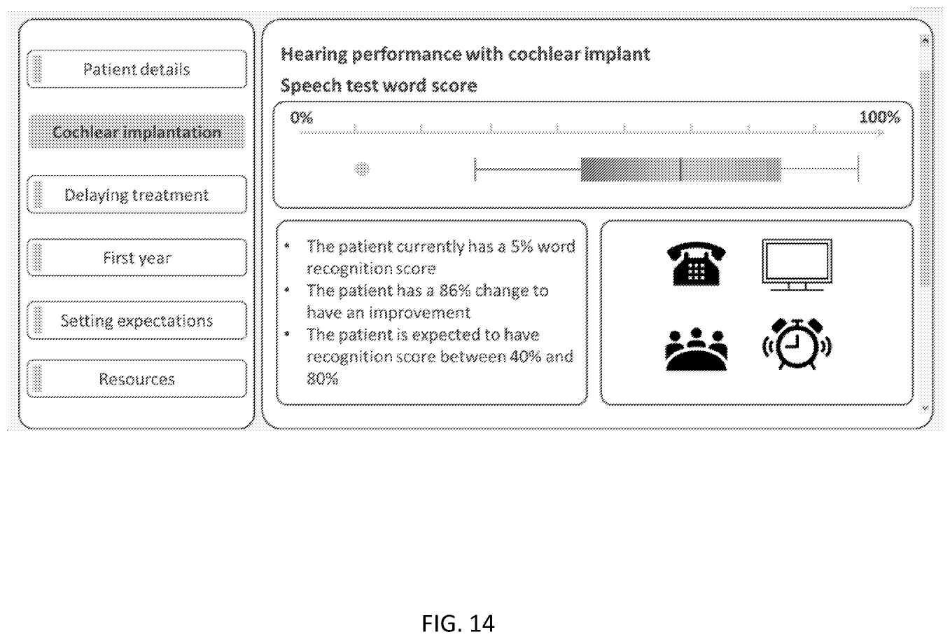

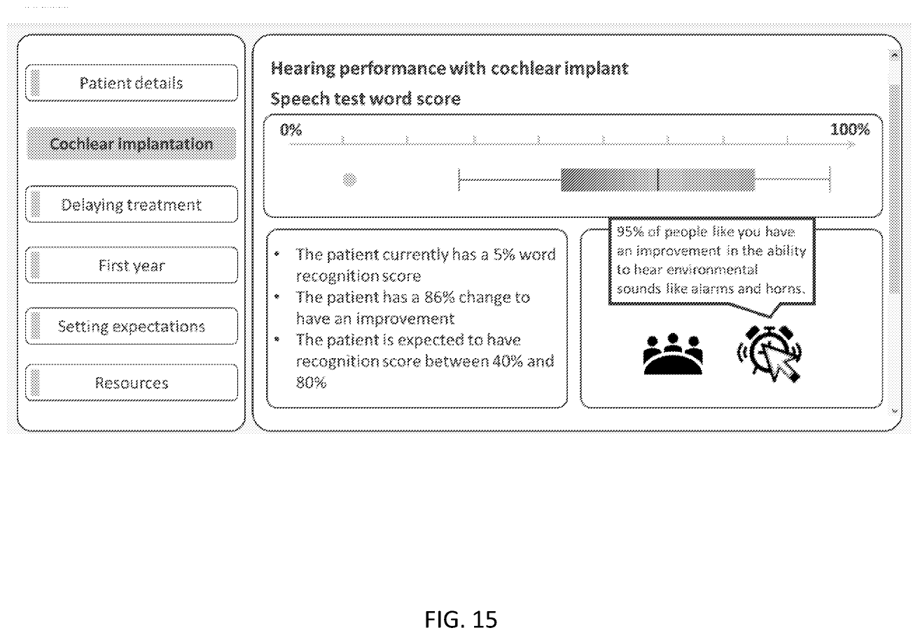

FIGS. 14 and 15 present an exemplary window that displays, by way of illustrative example only, cochlear implantation efficacy (outcomes) information personalised to the candidate. According to a clinical measure of hearing ability, the information conveyed is the candidate's estimated postoperative performance range juxtaposed with their current level of performance. There are written descriptions of the chance of a clinically significant improvement (a prediction according to methods 600 and 700) and the range of performance likely to be achieved. Additional information is displayed regarding the likely functional outcomes to be achieved associated with the range of clinical performance (again, a prediction).

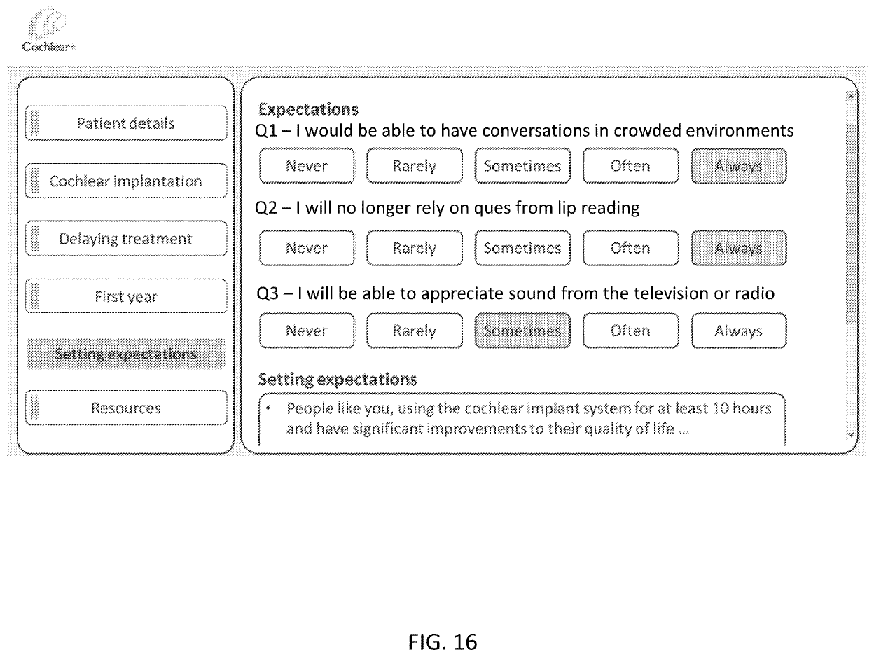

FIG. 16 presents an exemplary window containing one or more questionnaires that solicit information from the candidate of their expectations of the outcomes of cochlear implantation. This solicited information can be imported by the candidate and/or can be imported by the professional/clinician. The clinician/professional can use the processed/analyzed results of the questionnaires (analyzed per the teachings herein) to provide targeted counselling to address various beliefs of the candidate has. The goal of this counselling is to ensure that the candidate's expectations are reasonable or unreasonable, etc. Additional information can also be displayed such as prescribed activities or performative tasks that the candidate that the candidate should and/or must engage in to achieve their reasonable expectations of the treatment.

Accordingly, in an embodiment, the prediction of method 600 and/or method 700 is a prediction regarding a likely range of performance that is to be expected and/or a chance of achieving a clinically significant improvement. Note that by clinically significant it is meant something that can be measured and is more than a de minimus improvement. By way of example only and not by way of limitation, a candidate who is completely deaf could potentially hear garbled sound with a cochlear implant. That is an improvement but would not be considered a clinically significant improvement. Conversely, a person who can now engage in a conversation over a telephone link without text assistance, even haltingly and otherwise with difficulty, would be a clinically significant improvement over the complete inability to do so in the first instance without the cochlear implant. In an embodiment, a clinically significant improvement would be an improvement on an outcomes metric that would yield a degree of tangible benefit.

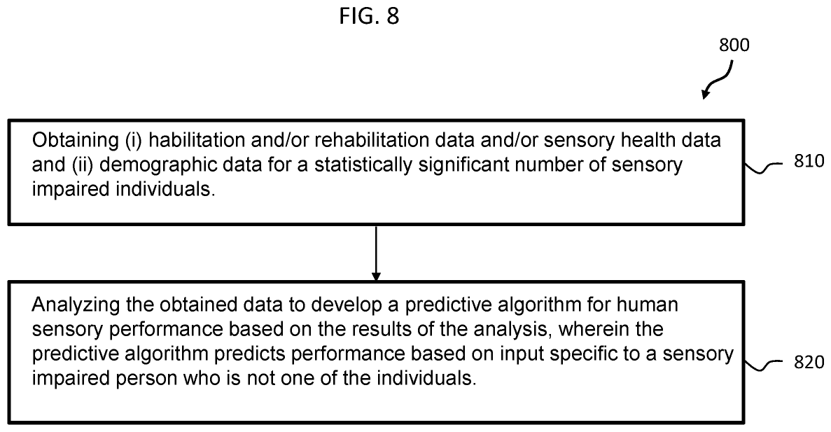

In view of the above, FIG. 8 presents an exemplary flowchart for an exemplary method, method 800. Method 800 includes method action 810, which includes obtaining (i) habilitation and/or rehabilitation data and/or sensory health data and (ii) demographic data for a statistically significant number of sensory impaired individuals. This can be sight impairment, hearing impairment, tactile impairment, balance impairment (herein, balance is a sensory impairment). It is noted that in another embodiment, method action 810 includes obtaining medical data for a statistically significant number of people who have a negative medical condition, such as, for example, high blood pressure, atrial fibrillation, etc. In an embodiment, the negative medical condition is a “rare” condition relative to other negative medical conditions (e.g., total hearing loss vs. cancer). This is where the machine learning/AI implementation can come into enhanced utility because the available data may not be sufficient enough to establish traditional statistical trends. For example, genetic anomalies leading to malformation/anomalous formation of the hearing organs can bar the use of traditional statistical trends. Certain types of cancer, such as vestibular schwannomas, squamous cell carcinoma, etc., are examples of conditions that could be better datamined and thus are more likely to be applicable to the trends (although this may not be the case).

The statistically significant number of sensory impaired individuals (or individuals having a negative health condition) corresponds to, for example, the data of the subjects detailed above by way of example only.

In an embodiment, the action of obtaining the data in method action 810 is executed by directly querying the sensory impaired individuals. In an exemplary embodiment, method action 810 is executed by obtaining data from organizations that have collected the data. Any way of obtaining the data that can enable the teachings detailed herein can be utilized in at least some exemplary embodiments.

In an exemplary embodiment, the demographic data can include any one or more of the demographic data detailed above or other demographic data. With respect to the habilitation and/or rehabilitation data, in this regard, this can correspond to performance on the clinical measures noted above, and/or can correspond to more generalized functional achievement measures. Some additional habilitation and/or rehabilitation data will be described below, but briefly, the habilitation and rehabilitation data can correspond to word recognition and/or word comprehension relative to that which was the case prior to beginning the treatment or otherwise utilizing a hearing prosthesis or a hearing device. Standardized testing can be included in the obtained data. In an embodiment, by way of example and not by way of limitation, some exemplary clinical measures can include:
An SSQ questionnaire
Nijmegen cochlear implant questionnaire
MOCA
Stroop test
Speech discrimination tests such as CNC, CVC, AzBIO, Austin
Phoneme discrimination tests such as LIT by NAL
Aided threshold test

Note that method action 810 also includes as an option the action of obtaining sensory health data. Here, this can correspond to the conditions of the candidate prior to treatment and/or after treatment. By way of example only and not by way of limitation, the severity of deafness of the candidate before obtaining a cochlear implant, such as, for example, the frequency range over which the candidate suffers hearing loss, the magnitude of that hearing loss for those frequencies, etc., how long that candidate suffered hearing loss the causes of the hearing loss, etc.

And note that in other embodiments, in a variation of method 800, there is a method that is not directly related to sensory elements, but instead related to other medical ailments, the habilitation and/or rehabilitation data can correspond to results of treatment compared to that which existed prior to treatment and instead a sensory health data, other types of health data can be obtained, such as the health data associated with the negative medical condition (the symptoms, the severity, the time that the candidate has been afflicted, etc.).

Method 800 includes method action 820, which includes analyzing the obtained data to develop a predictive algorithm for human sensory performance based on the results of the analysis, wherein the predictive algorithm predicts performance based on input specific to a sensory impaired person who is not one of the individuals. Again, in an embodiment, the action of analyzing the obtained data is executed using machine learning.

Briefly, in an exemplary embodiment, the individuals of method action 810 are hearing impaired individuals, the performance is performance with a sensory aid, the sensory aid is a hearing prosthesis (e.g., a cochlear implant), and the sensory impaired person is a hearing impaired person.

The predictive algorithm that predicts performance based on input specific to a sensory impaired person who is not one of the individuals can be what is used to execute method 600 or 700 above, where the input can be the data obtained in method action 610 noted above. The action of analyzing the obtained data can be executed using a machine network, neural network, DNN, to develop the predictive algorithm. Method action 820 is executed utilizing, for example, the DNN detailed above, although in other embodiments, any other type of machine learning algorithm or AI algorithm can be utilized in at least some exemplary embodiments. Consistent with the teachings further below, in an exemplary embodiment, the predictive algorithm is not focused on a specific feature. Instead, it utilizes a plurality of features that are unknown or otherwise generally represent a complex arrangement (if such can even be considered features in the traditional sense).

Still further, in some exemplary embodiments of method 800, consistent with the teachings detailed herein, the action of analyzing the obtained data machine-trains a system that results in the developed predictive algorithm. Also, still with respect to method 800, in an exemplary embodiment, of the obtained, at least a portion thereof is used, in the neural network, for training and at least portion thereof is used, in the neural network, for verification. Also, in this exemplary embodiment, the neural network develops the predictive algorithm by or transparently (sometimes, invisibly) identifying important features from the obtained data. Also consistent with the teachings herein, the predictive algorithm utilizes an unknowable number of features present in the speech data to predict hearing loss.

In an embodiment, the performance based on the results of the analysis is performance with a sensory aid device (a device of a specific type (e.g., a cochlear implant or an active transcutaneous bone conduction device), a specific make (Cochlear LTD cochlear implants) and/or a specific model (Nucleus 7™) and/or a specific design (cochlear implant having a 22 electrode array, or a 16 electrode array, or a 9 electrode array, etc.)).

There is a “contrary” embodiment, where the performance based on the results of the analysis is performance without a sensory aid device. Here, the performances what would happen if the person does not receive a sensory aid device. By way of example only and not by way of limitation, the performance can be how the person's speech will change one year, two years and/or three years and/or four years, etc., from a current date and/or a date in the past or in the future (where the dates in the past and/or the future can be correlated to an event, such as complete loss of hearing and/or the inability do here at frequencies above 1000 Hz by way of example). In an exemplary embodiment, this can have utilitarian value with respect to “showing” a candidate what will happen if the candidate does not take one a given treatment. This can be used for comparison purposes to compare the results of what would happen without treatment to what would happen with treatment.

Concomitant with the teachings above, in an embodiment, the action of analyzing the obtained data machine-trains a system that results in the developed predictive algorithm. As noted above, in some embodiments, the use of machine learning includes calculating coefficients, parameters, thresholds and/or weights of models by minimizing a cost function.

In some embodiments, at least a portion of the obtained data is used, in a neural network (or whatever AI system is used), for training and at least a portion thereof is used, in the neural network, for verification. Also, in this exemplary embodiment, the neural network develops the predictive algorithm by transparently (sometimes, invisibly) identifying important features from the obtained data. Also consistent with the teachings above, the predictive algorithm utilizes an unknowable number of features present in the speech data to predict the output.

With respect to the unknown/transparent features, these can be features that are not related to a simple data description.

In an exemplary embodiment, the machine learning develops the predictive algorithm by internally identifying important features from the obtained data. For example, in implementing method 800, action 820 is executed utilizing, for example, the DNN detailed above, although in other embodiments, any other type of machine learning algorithm can be utilized in at least some exemplary embodiments. Consistent with the teachings detailed above, in an exemplary embodiment, the predictive algorithm is not focused on specific feature. Instead, it utilizes a plurality of features that are unknown or otherwise generally represent a complex arrangement (if such can even be considered features in the traditional sense). Thus, the predictive algorithm utilizes a complex arrangement number of features present in the habilitation and/or rehabilitation data to predict the human performance.

In an embodiment, method 800 further includes curation of the obtained data to remove data having records that are sparely populated and/or are low quality (relatively low quality). In an exemplary embodiment, these are relative characteristics of the data relative to other portions of the data. In an exemplary embodiment, a threshold for determining or otherwise classifying such to be the case can be set at the outset or during data development and/or upon completion of data acquisition. With respect to this latter regime, it is possible that additional data will have to be obtained because sufficient amounts the data will not be usable or otherwise must be manipulated to an extent that becomes unfeasible.

In an exemplary embodiment, method 800 further includes the action of filling in missing data using patterns and/or relationships within the obtained data to obtain filled in data, which filled in data includes the obtained data. In an exemplary embodiment, this action of filling in missing data is executed by a product of the neural results of machine learning, or other form of artificial intelligence system. In an exemplary embodiment, statistical software can be utilized to fill in missing data. Any device, system, and/or method that can enable the action of filling in missing data that will have utilitarian result can be utilized providing that the art enables such. The method can further include the action of analyzing the filled in data to develop the predictive algorithm for human performance with the sensory aid based on the results of the analysis, wherein the predictive algorithm predicts performance with the sensory aid based on input specific to the sensory impaired person who is not one of the individuals, wherein the action of analyzing the filled in data includes the action of analyzing the obtained data.

In a specific implementation of method 800, where, for example (but not limited to) the performance is performance with a sensory aid, and the individuals are hearing impaired individuals, and the hearing prosthesis is a cochlear implant, the action of analyzing the obtained data, method 820, includes (i) transforming preoperative factors (or pre sensory aid use factors) included in the obtained data using principle component analysis and/or independent component analysis and/or (ii) transforming information in the obtained data into numerical forms. Still further, in a specific implementation of method 800, again with the just-noted conditions precedent by way of example and not by way of limitation, the action of analyzing the obtained data, method 820, includes (i) identifying preoperative factors and/or transformed factors of the individuals that correlate to a predictable outcome using the cochlear implant and/or (ii) identifying preoperative factors and/or transformed factors of the individuals that casually relate to a predictable outcome using the cochlear implant.

Returning back to methods 600 and 700, in an embodiment, the action of analyzing the obtained data (method actions 620 and 720) or otherwise an action of analyzing the obtained data is executed using a code from and/or of a machine learning algorithm to develop the output.

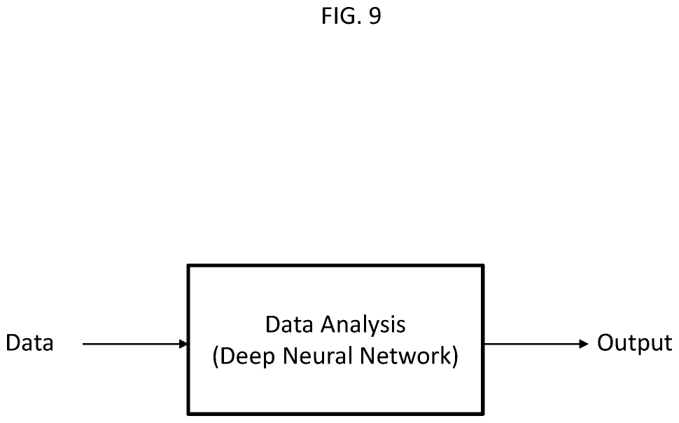

FIG. 9 depicts an exemplary conceptual schematic of an example of a portion of method 600, where the obtained data is the input into a system that utilizes a trained DNN or some other trained learning algorithm (or the results thereof—the code of a machine learning algorithm as used herein corresponds to a trained learning algorithm as used in operational mode after training has ceased and code from a machine learning algorithm corresponds to a code that is developed as a result of training of the algorithm-again, this will be described in greater detail below), and the output is information that can be used to develop a prediction or is the prediction.

It is noted that in at least some exemplary embodiments, it is not the obtained data that is provided directly to the learning algorithm. Instead, one or more feature extractions of the data are calculated and provided as inputs to the learning algorithm.

Any data that is based on the data from the person/about the person can be utilized in at least some exemplary embodiments that can enable the teachings detailed herein can be utilized as input into the signal analysis algorithm.

In an embodiment, information such as age, data relating to the onset of deafness (how long ago, how long since birth, type, suddenness, etc.), the gender of the candidate and the raw speech of the candidate, and/or other data is inputted into the DNN.

In an exemplary embodiment, the more independent information containing specific characteristics of the person provided to the learning model, the more accurate the prediction. To be clear, embodiments include systems with a range of different inputs. The “output” can be one or more feature extractions.

Note further that in some embodiments, some or all of the data is pre-processed data and not “raw data” that is input into the DNN. Any data that can enable the DNN or other machine learning algorithm to operate can be utilized in at least some exemplary embodiments.

Note that the embodiments are used to predict things other than hearing health measures and other than hearing benefit outputs. In some embodiments, the predictions are not either of these.

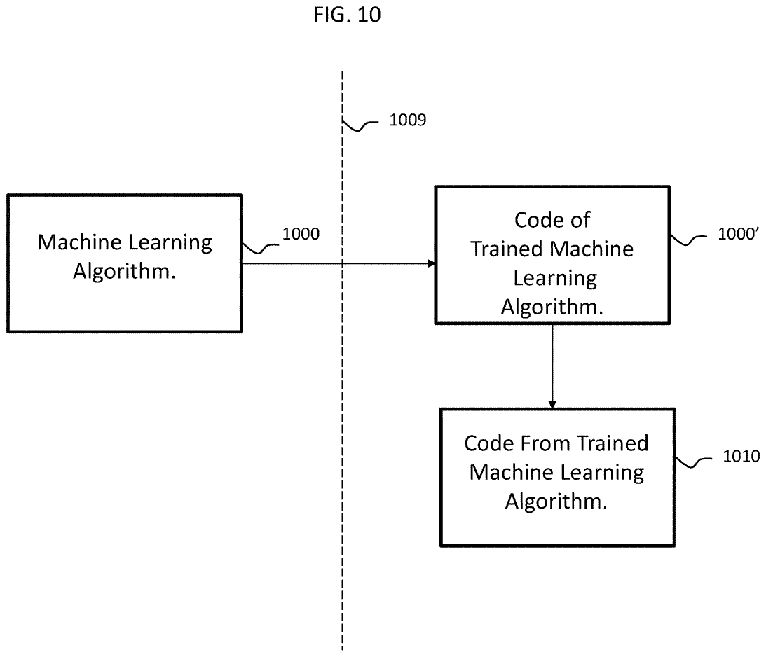

As noted above, the methods herein can utilize code from a machine learning algorithm and/or a code of a machine learning algorithm. In this regard, the code can correspond to a trained neural network (the latter). That is, as detailed herein, in some embodiments, a neural network can be “fed” statistically significant amounts of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. FIG. 10 depicts by way of conceptual schematic exemplary “paths” of obtaining code utilized in at least some of the methods herein. With respect to the first path, the machine learning algorithm 1000 starts off untrained, and then the machine learning algorithm is trained and “graduates” by symbolically crossing the line 1099, or matures into a usable code 1000′—code of trained machine learning algorithm. With respect to the second path, the code 1010—the code from a trained machine learning algorithm—is the “offspring” of the trained machine learning algorithm 1000′ (or some variant thereof, or predecessor thereof), which could be considered a mutant offspring or a clone thereof. That is, with respect to the second path, in at least some exemplary embodiments, the features of the machine learning algorithm that enabled the machine learning algorithm to learn may not be utilized in the practice of some of the methods herein, and thus are not present in version 1010. Instead, only the resulting product of the learning is used.

In an exemplary embodiment, the code from and/or of the machine learning algorithm utilizes non-heuristic processing to develop the data regarding hearing loss. In this regard, the system that is utilized to execute method 600 or 700, for example, takes the data and extracts fundamental signal(s) therefrom, and uses this to produce the output. By way of example only and not by way of limitation, the system utilizes algorithms beyond a first-order linear algorithm, and “looks” at more than a single extracted feature. Instead, the algorithm “looks” to a plurality of features. Moreover, the algorithm utilizes a higher order nonlinear statistical model, which self learns what feature(s) in the input is important to investigate. As noted above, in an exemplary embodiment, a DNN is utilized to achieve such. Indeed, in an exemplary embodiment, as a basis for implementing the teachings detailed herein, there is an underlying assumption that data that enables the prediction to be made are too complex to be specified, and the DNN is utilized in a manner without knowledge as to what exactly on which the algorithm is basing its prediction/at which the algorithm is looking to develop its prediction. Practically, herein, a probabilistic model will output a probability of a value of an event occurring, whereas a statistical model predicts the value itself. For example, a Bayesian interference vs. an ordinary least squares regression.

In at least some exemplary embodiments, the DNN is the resulting code used to make the prediction. In the training phase there are many training operations algorithms which are used, which are removed once the DNN is trained.

More generally, according to the teachings detailed herein, generic features are utilized. In some embodiments, no specific feature is utilized, or at least with respect to executing method 600 or 700 (or any of the other methods detailed herein), there is no specific feature of the speech and/or the biographic data that is looked at. Thus, a non-heuristic processing method is utilized. To be clear, in at least some embodiments, the specific features utilized to execute the methods are not known (in some instances, they are not otherwise described), and one does not need to care as to what specific features are utilized. In at least some exemplary embodiments, a learning system is utilized which arbitrarily picks features within the input into the system in an attempt to “learn” how to make the predictions (or at least develop information usable to make the predictions) and once the system learns how to predict or develop the information that can be used to make the prediction, it performs accordingly.

To be clear, in at least some exemplary embodiments, the trained algorithm is such that one cannot analyze the trained algorithm or the resulting code therefrom to identify what signal features or otherwise what input features are utilized to make the output. In some embodiments, it is never known what the system has identified as important at the time that the systems training is complete. The system is permitted to work itself out to train itself and otherwise learn to make the output.

Embodiments include systems that implement at least some of the methods/functions disclosed herein. In an embodiment, there is a computer system that includes an input subsystem (e.g., including the input component(s) of the devices detailed above, a touch screen, a keyboard, a microphone, a mouse and the accompanying computer screen, a USB port or an internet connection or a Bluetooth connection and the associated hardware and software) configured to receive input regarding information about a hearing impaired person. As can be understood from the just detailed exemplary components, the input subsystem need not directly interface with a human (USB port vs. a keyboard and mouse and/or a touch screen), but in some embodiments the input subsystem does so directly interface with a human.

The computer system further includes an output subsystem (e.g., including the output component(s) of the devices detailed above, a touch screen, a keyboard, a microphone, a mouse and the accompanying computer screen, a USB port or an internet connection or a Bluetooth connection and the associated hardware and software). Any device, system, and/or method that can enable the input of any part or all of the data that is utilized by the teachings detailed herein that has utilitarian value can be utilized in at least some exemplary embodiments, and the same is the case with respect to any device system and/or method that can enable the output of any part or all of the information that is disclosed herein and/or variations thereof that has utilitarian value can be utilized in at least some exemplary embodiments.

In embodiments, there is an artificial intelligence subsystem interposed between the input subsystem and the output subsystem, wherein the system is configured to predict, with the use of the artificial intelligence subsystem, hearing performance of the hearing impaired person resulting from use of a hearing device and/or resulting from lack of use of the hearing device based on the input into the input subsystem. In an embodiment, the artificial intelligence system is a neural network that is at least a partially taught neural network (which means that it can be a fully taught neural network). In an embodiment, the artificial intelligence system is a neural network that is a partially taught neural network that is trainable with feedback provided through the input subsystem or another subsystem of the system.

With regard to the prediction of hearing performance of the hearing impaired person resulting from use of a hearing device and/or resulting from lack of use of the hearing device based on the input into the input subsystem, the former can correspond to any of the above predictions or other predictions that can be enabled by the teachings detailed herein and have utilitarian value. For example, the above detailed functional activities at which the hearing impaired person can participate in if the person utilizes the hearing device can be the prediction. Scores on standardized speech or hearing tests, etc., can be the prediction. The ability to hear at certain frequencies can be the prediction. Conversely, with respect to the prediction resulting from lack of use of the hearing device, this can correspond to a prediction associated with how the person's speech will change over time, such as how his or her voice will change over time (because for example the person will not be able to hear himself or herself speak, or will have a very difficult time hearing himself or herself speak, or will hear only some frequencies and not others or at least meaningfully hear some frequencies and not meaningfully hear others, etc.). This can correspond to a prediction associated with how well (or poorly) the person will perform on standardized speech tester hearing tests, etc. the idea here is that the system can be utilized to show the medical utilitarian value of the hearing device, and can, in some instances, convince and otherwise hesitant person that he or she should utilize this hearing device by showing meaningful data that is personalized to the person, as opposed to general data or general performance metrics from generalized studies, even if such are somewhat tailored to the individual person. Note that this is not a hearing loss prediction. This is a hearing performance. How well the person will hear based on a recognition that hearing loss is going to happen. Put another way, a method of using the system would have hearing loss prediction “baked into” the method before making the prediction. Indeed, in an embodiment, the prediction can be a prediction of hearing performance of the hearing impaired person resulting from lack of use of the hearing device with continued deterioration of the person's hearing sense in the future.

In an embodiment, the system is configured to automatically transform the input into numerical form. This can be executed using a computer chip or a logic circuit or electronics or software or a processor, that is programmed to take the input and transform the input. In some embodiments, the input subsystem is configured to execute this functionality. Thus, the input subsystem can be more than just a mouse and computer screen and keyboard, etc. Embodiments include an input subsystem that includes a processor and/or software and/or firmware and/or hardware and/or a computer chip or a logic circuit otherwise electronics that is specifically designed and configured to execute one or more of the functionalities of the input subsystem detailed herein.

The artificial intelligence subsystem is configured to, using the numerical form, automatically produce an estimated outcomes measure for the person and the output subsystem is configured to transform the estimated outcomes measure into an output forecast corresponding to the prediction of the hearing performance of the hearing impaired person resulting from use of a hearing device.

In an embodiment, the output subsystem provides the output forecast in a natural language message form. In an embodiment, the natural language messaging can be in the form of: (i) a description of the likely range of performance to be expected; (ii) a description of the chance of achieving a clinically significant improvement; (iii) information regarding prescribed activities that the candidate/person should (or must) engage in to achieve the prediction such as for example the amount of time using the cochlear implant system and/or the sound environments where the candidate should use the device; and/or (iv) information regarding the functional activities that the person/candidate more likely than not will be able to engage in, such as corresponding to the predicted clinical measure.

The system can be configured to transform the input into numerical form, the artificial intelligence subsystem can be configured to, using the numerical form, produce an estimated outcomes measure, produce a prediction interval and produce a probability density function and the output subsystem can be configured to transform the estimated outcomes measure, the prediction interview and the probably density function into an output forecast corresponding to the prediction of the hearing performance of the hearing impaired person resulting from use of a hearing device, which output forecast provides a range of performance to be expected and chances of achieving a clinically significant improvement with the use of the hearing device.

As can be seen, embodiments of the output subsystem can be more than just a “dumb” computer screen or the like. Embodiments include an output subsystem that includes a processor and/or software and/or firmware and/or hardware and/or a computer chip (herein a computer chip also corresponds to a plurality of such, interconnected with a motherboard, etc.) or otherwise electronics that is specifically designed and configured to execute one or more of the functionalities of the output subsystem detailed herein. Any disclosure herein of software corresponds to an alternate disclosure of a computer chip or a logic circuit or electronics.

As noted above, in an embodiment, the output subsystem provides forecasted results on one or more different clinical speech tests based on the prediction. That said, different clinical speech tests have different levels of difficulties to discern the extent of the hearing ability of the subject/the person at interest to overcome floor and ceiling effects. In this regard, there are tests that “max out” with respect to the ceiling effects, where for example the top score does not capture all of the hearing performance or otherwise does not convey the full story in a clinical manner of the persons hearing ability. Conversely, these tests have floors where the lowest score might not adequately or clinically convey how poorly the person hears. But it could be that the data input is sufficient to develop a forecast for performance on some tests and not others, where the tests that can be forecasted have the ceiling/floor effects, and the tests that cannot be forecasted are the ones where there will be no ceiling/floor effect. That is, various modelling approaches may not be performative in directly predicting the more difficult speech tests and/or may not yield as accurate results for the more difficult speech test relative to the easer/less difficult speech tests.

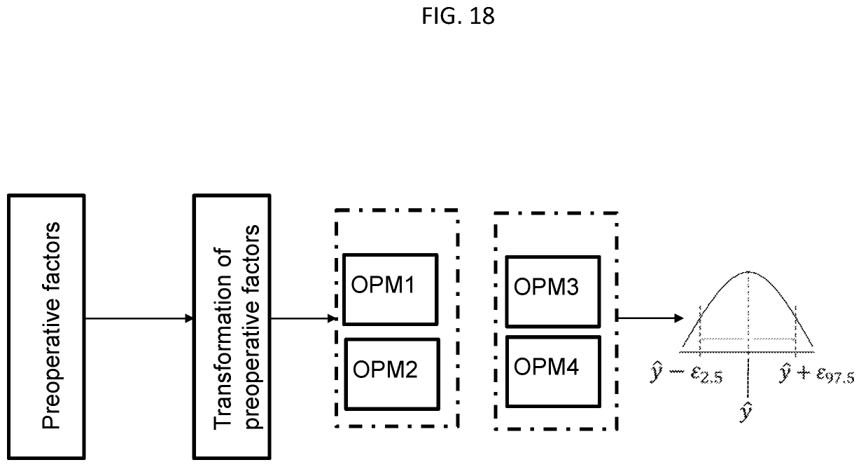

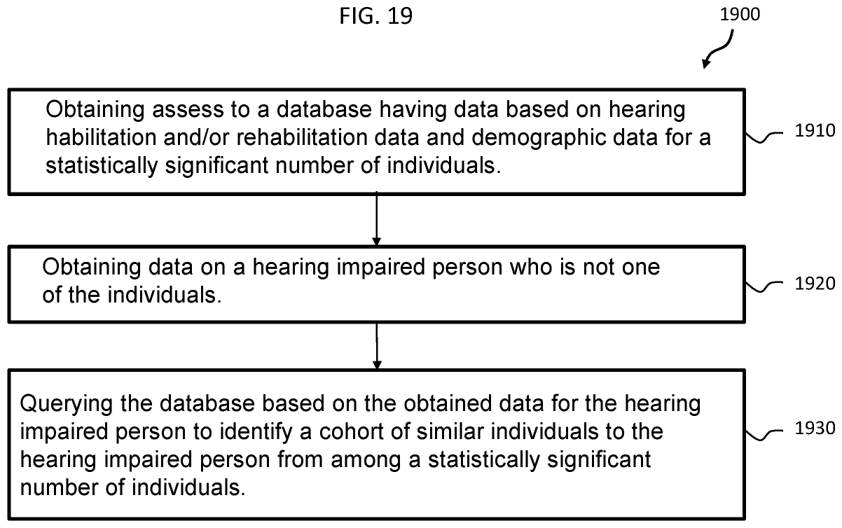

In some embodiments, the metrics of different speech tests that are applicable to the teachings herein are correlated in a utilitarian manner, including relatively highly correlated. More difficult speech test results are predicted by a chain of prediction models (which can include two prediction models. This is shown by way of example in FIG. 18, where a plurality of outcomes prediction models (OPM1 to OPM4) are used, where at least some of the models predict the results of different tests, where the prediction/results from one model is used by the next model to form a prediction/produce results therefrom. That is, the prediction of the previous model is used as an input into the prediction the subsequent model, along with preoperative factors/pretreatment factors/some or all of the data obtained by the system/input into the system. The chain of prediction models is sequenced either by an optimization process such that information generation through the chain is maximized in at least some embodiments.

Thus, in an embodiment, the artificial intelligence subsystem is configured to, based on the input, develop a first prediction of a result of first speech test for the person using the hearing device, and based at least in part on the first prediction and the input, develop a second prediction of a result of a second speech test for the person using the hearing device, wherein the second speech test is more difficult than the first speech test.

And it is noted that in some embodiments, the concept of the chain is not limited to application to hearing tests. This can be utilized for other clinical measures. Any clinical measured that can be estimated or otherwise predicted utilizing the chain technique that has utilitarian value can be utilized in at least some exemplary embodiments, providing that the art enables such. Thus, in an embodiment, the artificial intelligence subsystem applies a chain of prediction modes to predict hearing performance of the hearing impaired person resulting from use of the hearing device based on the input, wherein subsequent model(s) in the chain use result(s) of prior model(s) to overcome floor and/or ceiling effects of the prior model(s).

Embodiments can include a device, such as a computer, that includes an input suite configured to receive input relating to a sensory impaired person (or a person who suffers from a negative medical condition) and electronics configured to analyze data based on input into the input suite (the data based on input can be the input, or it can be modified data). In an embodiment, the electronics includes a prediction model that is used by the device to automatically predict performance capabilities of the sensory impaired person (or a person who suffers from a negative medical condition) after receiving a sensory aid (or after beginning receipt of treatment) and using such for a given period of time (or continuing treatment for a given period of time) based on the input into the input suite. This can correspond to any of the devices detailed above having this functionality, which device includes, for example, a chip that is the product of or result of machine learning that includes the prediction model.

In an exemplary embodiment, the sensory aid can be a vision device or a hearing device, such as a consumer-electronics hearing assistance device, or can be a hearing prosthesis, such as a conventional hearing aid, or a bone conduction device (active transcutaneous, passive transcutaneous and/or percutaneous) or a middle-ear implant, or a cochlear implant by way of example. Some of these will be recognized to the prostheses that requires some form of surgery, while others may not necessarily require such. Any reference herein to a postoperative date corresponds to a disclosure of a post beginning of use date for any of those prostheses.

While the embodiment just described presents a device that is utilized to evaluate a sensory impaired person, it is noted that, consistent with the statement above that disclosures directed to certain features corresponds to a disclosure of other features in the interests of textual economy, the above device can be directed towards evaluating people with other types of negative health conditions, such as, for example, any of those detailed herein.

In an embodiment, the just-detailed device can have, in addition to this, or alternatively, a cohort comparator that is used by the device to automatically identify a cohort of people with statistically significantly similar and/or the same pre-sensory aid use factors as the sensory impaired person. The concept of the cohort comparator is different from the prediction model. By way of example only and not by way of limitation, the cohort comparator identifies a group of live or formerly alive humans that have characteristics similar to the sensory impaired person, whereas the prediction model is predictive of a result or outcome. A human can analyze the identified cohort and then make judgements based thereon. A prediction can be made, by the human. This as opposed to the model making the prediction. That said, embodiments include a device and/or system that makes a prediction based on the output of the cohort comparator.

With the availability of large datasets (i.e., big data) with many subjects' records (for example, more than the threshold needed to develop a utilitarian prediction model using the AI/machine learning, but not necessarily more than what such model uses or could use, because the model can get better potentially with more records, but it could work satisfactorily with fewer records) with preoperative factors/pre-treatment factors data and postoperative/post beginning of treatment outcomes data (e.g., post beginning of use of a sensory aid, such as a hearing device or a vision device, such as a consumer-products hearing enhancement device, or a hearing prosthesis, such as a conventional hearing aid, or a cochlear implant or a bone conduction device or a middle-ear implant, etc.) it is possible to directly compare, for example, sensory prosthesis candidates, such as bone conduction implant candidates and/or cochlear implant candidates with a cohort of people with similar and/or the same preoperative/pretreatment factors. The cohort is generated when the cohort comparator is run rather than being predetermined. Thus, embodiments include the establishment of a bespoke cohort of people. Thus, in an embodiment, a bespoke cohort of people is developed for each new hearing impaired person evaluated by the device. Based on a set of utilitarian preoperative/pretreatment factors (e.g., age, duration of ailment (e.g., hearing loss or sight loss or balance loss), aetiology, clinical measures, etc., and any of those detailed herein with respect to the obtained data providing that such has utilitarian value in the art enables such), filters are constructed by the device, automatically, to identify and isolate a cohort of statistically similar people. The filters can be substantiated by creating upper and lower limits (less than the threshold of clinical significance difference) around one or more of the available preoperative factors. Thereafter, the filters can be translated into a database query and postoperative/post beginning of treatment clinical measures and functional outcomes data comprising many subjects is retrieved. This data from the many people can be directly compared against the candidate's preoperative/pretreatment clinical measures and functional ability. The comparison can be processed by data processing algorithms to highlight statistically, probabilistically, linguistically and/or visually various pieces of information that have utilitarian value with respect to counseling the candidate, for example: (i) the range and/or distribution of postoperative/post beginning of treatment clinical measures (e.g., such as those detailed above) of the subjects of the cohort/members of the cohort (measures for any of the clinicals detailed herein, for example), which can be temporally based (results for post beginning of treatment at 7, 14, 21, 28, 31, 45, 60, 75, 90, 120, 150, 200, 250, 300, 350, 400, 450, 500, 600, 700, 800, 900, 1000, 1250, 1500, 1750 or 2000 days or more or any value or range of values therebetween in 1 day increments); (ii) a likelihood that the candidate will improve (this can be determined by, for example, integrating the probability density function of the cohort's post operative/post beginning of treatment clinical measures from where the candidate is currently performing to the maximum values of the clinical measures); (iii) a likelihood of the different functional activities that the candidate could engaging in if the candidate were to commence treatment, such as receive the sensory aid and use such, such as receive the cochlear implant and use such (for example, 80% of the cohort engages in regular discussion with others in quite environments (regularly and/or in a non-hesitant and/or non-apprehensive manner, for example)); (iv) relative to the date of the beginning of treatment, such as implantation of the implantable portion of a hearing prosthesis and initial switch-on of the candidate's sound processor, estimated dates of periods of time of the achievement of certain ranges and/or values of clinical outcomes measures and/or functional outcomes; and/or (v) a description of how the cohort engages in their treatment, such as uses their sensory aid, such as uses their cochlear implant device post switch-on such as averages and ranges of the daily amount of time using the device and the amount of time using the device in different sound environments (for example, time in speech and conversations, time in noisy speech and conversations, time in music, time in quiet, etc.) it is briefly noted that in some embodiments, the predictions established by the model can predict any one or more of these highlighted features. And note that in some embodiments, any one or more of the predicted features that are predicted by the model can, in some embodiments, be highlighted utilizing the data based on the cohort comparator. Embodiments include a device that is configured to provide data that is configured to be used to present, and/or is one or more or all of (i) to (v). In an embodiment, the probability density function has the numerical range on the x-axis and the probability density of the y-axis. The integration between two different values of x give one a probability. For a measure of outcomes, one adds what would be considered a clinical significant improvement to the initial preoperative value and integrates beyond that point to infinity. This will give the probability or the likelihood that the candidate will receive a clinically significant improvement.

In view of this, an exemplary expanded method 1900 and/or method 2000 includes the action of, based on the retrieved measures and/or functional outcomes, presenting to the person (and in an embodiment, the device and/or system detailed above can be configured to provide data that is configured to be used to present, and/or is at least one of the following): a range and/or distribution of postoperative clinical measures of the subjects of the cohort; a likelihood that the person will improve by integrating a probability density function of the cohort's post operative clinical measures from where the person is currently performing to the maximum values of the clinical measures; a likelihood of different functional activities in which the person could engage if the person were to receive the cochlear implant; relative to a date of first use of the person's hearing device, estimated dates and/or periods of time of achievement of certain ranges of clinical outcomes measures and/or functional outcomes; and/or a description of how the subjects of the cohort use their hearing device.

With respect to the device detailed above, in some embodiments, the device includes a data analysis algorithm that highlights statistical, probabilistic, linguistically and/or visually one or more cohort informational datapoints of the cohort of subjects. But in some embodiments, the output from the cohort comparator can be used by a human to develop this data.

The above-noted identification of cohort concept lends itself to an exemplary method. In this regard, FIG. 18 shows an exemplary method, method 1800, that includes method action 1910, which includes obtaining access to a database having data based on hearing habilitation and/or rehabilitation data and demographic data for a statistically significant number of individuals (e.g., any of the numbers detailed herein). The method also includes method action 1920, which includes obtaining data on a hearing impaired person who is not one of the individuals. This can be executed according to any of the methods and/or by any of the devices and/or systems herein. Method 1900 also includes method action 1930, which includes querying the database based on the obtained data for the hearing impaired person to identify (automatically identify) a cohort of similar individuals to the hearing impaired person from among a statistically significant number of individuals.

In an embodiment, the statistically significant number of individuals of the database is a number that enables the identification of the cohort of similar individuals. In an embodiment, the identified cohort is a cohort that has never been identified before. And consistent with the teachings above, the identified cohort is a bespoke cohort and the statistically significant number of individuals are recipients of a surgically implanted hearing prosthesis. In an embodiment, the identified cohort is a cohort that has never been identified before by the entity executing method 1900.

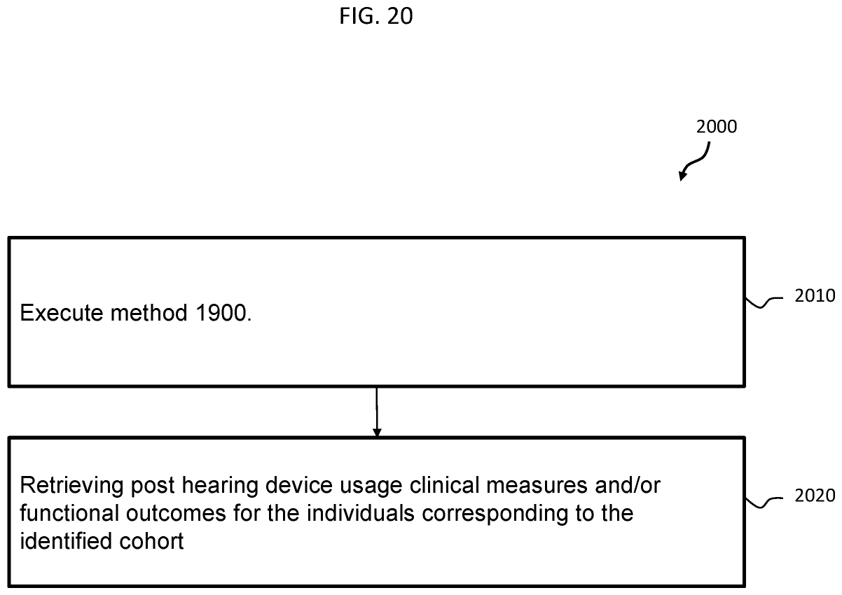

FIG. 20 shows another method, method 2000, which includes method action 2010, which includes executing method 1900. Method 2000 includes method action 2020, which includes retrieving post hearing device usage clinical measures and/or functional outcomes for the individuals corresponding to the identified cohort, and in some embodiments, this is done automatically, such as by any of the systems or devices herein. In an embodiment, the method is expanded to include counseling the hearing impaired person on the likely results of him or her obtaining a sensory aid, such as a hearing prosthesis, such as a cochlear implant, consistent with the teachings herein.

In an embodiment, method 1900 and/or method 2000 is expanded to include the action of automatically, based on the retrieved measures and/or functional outcomes, developing a plausible scenario for the individual for hearing habilitation and/or rehabilitation using a type and/or design of hearing prosthesis, wherein the data of the database includes data based on habilitation and/or rehabilitation data and demographic data for a statistically significant number of individuals who use the type and/or design of hearing prosthesis. The hearing prosthesis is a cochlear implant in some embodiments, and thus the habilitation and/or rehabilitation data and demographic data for a statistically significant number of individuals is for individuals who have received a cochlear implant.

The number can be any of those detailed herein. Also, this action can be executed automatically, in some embodiments.

In an embodiment, method 1900 and/or method 2000 is expanded to include the action of automatically generating filters based on the obtained data for the hearing impaired person, wherein the action of querying the database is executed based on the automatically generated filters. Also, the methods can be expanded so that the method(s) include, based on the retrieved measures and/or functional outcomes, providing a forecast of hearing habilitation and/or rehabilitation using a type and/or design of hearing prosthesis, wherein the data of the database includes data based on habilitation and/or rehabilitation data and demographic data for a statistically significant number of individuals who use the type and/or design of hearing prosthesis. And in an embodiment, the devices and/or systems herein include electronics that include a prediction model and the prediction model includes one or more of an outcome submodel, a trajectory of perform submodel and/or risk of delay submodel.

Embodiments include identifying rates and/or the nature of a progression of sensory loss, such as hearing and/or vision and/or balance loss based on various causes, severity and/or duration of the sensory loss. Models are constructed to do so in an exemplary embodiment. These models can be utilized and/or the identification of rates can be utilized to show the risk of delay in at least some exemplary embodiments.

Further, the device can be configured to utilize an unsupervised clustering algorithm to identify groups of subjects with statistically similar (significantly similar) patterns of progression of hearing sensory loss (obtained from the data obtained about the person with sensory impairment) and thus exclude subjects that do not have similar patterns. (This can be done using, for example, large datasets of subjects' demographics, health in general and sensory health (e.g., hearing, vision, etc.) in particular (both can be used) and history of pre treatment/post beginning of treatment clinical measures and/or any of the other factors detailed herein.) And to be clear, with respect to the embodiments that identify or otherwise develop cohorts, such development and/or identification can be achieved by excluding subjects/members of the database based on statistically significant dissimilarities or even non-statistically significant dissimilarities. Both techniques can be implemented to develop the cohorts, whether for risk of delay or for the other cohort based teachings herein. Also, in an embodiment, the device is configured to, for respective groups of subjects, identify factors to form/establish respective signatures of the respective groups. This can be done automatically using computer models and/or electronics specifically designed to do so. In an embodiment, this can be done by a person/manually. The signatures can be stored and used in the future. In an embodiment, the respective groups have respective characteristic hearing loss patterns over time, and in an embodiment, the characteristic hearing loss patterns can be distinguished from each other in a meaningful manner by one of skill in the art. And in at least some exemplary embodiments, the device is configured to compare, automatically, data based on the received input to identify a matched group from the respective groups based on the respective signatures. But note that in some embodiments, the signatures are not necessarily utilized. Instead, the data can be evaluated in totality and/or at least in a larger manner than that which results from the utilization of signatures for example, and the groups can be identified therefrom or otherwise the matched groups can be developed from this larger set of data. Computer algorithms can be utilized. Artificial intelligence or otherwise the products thereof can be utilized to perform this analysis, even though the ultimate groups are based on actual data. The device is configured to adjust, automatically, the characteristic hearing loss pattern of the matched group in accordance with the person's current and/or historical clinical measures. For example, by way of example, this adjustment can show what would happen without the implementation of the treatments and what would happen with the implementation of the treatments. In an embodiment, the device is configured to provide information based on the adjusted characterized hearing loss pattern. This information can be presented in natural language, or can be presented in healthcare professional language that the healthcare professional can “translate” to language that is understandable by the sensory impaired person. This is the case with all of the embodiments herein. Any disclosure herein of utilizing natural language corresponds to a disclosure where the output is not natural language or otherwise the output is language that must be interpreted or should be interpreted for the layman to better understand.

Note also that the risk of delay can be directed towards scenarios that do not necessarily present as a baseline no treatment. In an exemplary embodiment, two different treatments could be compared to one another. By way of example only and not by way of limitation, the risk of delay or otherwise the comparisons can be between, for example, what happens if a recipient or a candidate continues to utilize a conventional hearing aid instead of obtaining a cochlear implant or an active transcutaneous bone conduction device or some other bone conduction device for that matter or a middle-ear implant for example. Indeed, in an embodiment, the “pre-treatment” data can correspond to the data that results from the candidate utilizing that conventional hearing aid. This can be the baseline from which the risk of delay is demonstrated. Accordingly, risk of delay models are not necessarily directed to how the hearing loss will progress untreated. These can be directed to how the hearing loss will progress in a treated first manner (or the effects thereof), and then how that hearing loss (or the effects thereof) would progress if treated in another manner different from the first manner). Moreover, the treatments can be difference in degrees, not just kind. By way of example only and not by way of limitation, the person can be utilizing a generic conventional hearing aid that was obtained without a prescription for example. This conventional hearing aid could be a hearing aid that was obtained without input from an audiologist, or at least without input based on an audiogram or the like. Conversely, the methods herein can show what would happen with a conventional hearing aid that is based on a prescription, including one that has adjustments for different frequencies, because, for example, the candidate can hear better at some frequencies versus others. This is something that may not exist with the conventional generic hearing aid which hearing aid was obtained without a prescription. Accordingly, embodiments herein can compare the results of what would happen with a more sophisticated or otherwise a targeted application of a hearing aid, such as on based on a prescription, versus the continued use of the current hearing aid. Note further that the differences in prescription can be shown. By way of example, the existing hearing aid may be based on a prescription. Hearing aids can be expensive or otherwise cost money, and the risk of delay models can be utilized to show that it is worthwhile for the candidate to spend more money or otherwise have the hearing aid adjusted.

And note that the above comparisons are not limited to the use of risk of delay models. Indeed, irrespective of comparisons, any disclosure herein related to a pretreatment and/or pre-use of a medical device can correspond use of a medical device, such as a type of sensory supplement device, where the post beginning of treatment/post beginning of use can be the point where the new, different medical device is started to be used. Again, this can correspond to the transition of a conventional hearing aid (pretreatment) to a cochlear implant. Accordingly, in a variation of method 600 and/or 700, the prediction is a risk of delay of use of the sensory supplement device by the human vs use of another sensory supplement device by the human (delay of cochlear implant vs. continued use of a conventional hearing aid, for example).

In view of the above, in an embodiment, where, for example, the device includes the model, the model is a product of and/or resulting from machine learning that is used by the device to predict performance capabilities of the sensory impaired person after receiving the sensory aid and using such for the given period of time based on the input into the input suite. In an embodiment, the prediction can correspond to any one or more of the predictions detailed herein and the prediction can be made according to any one or more of the methods detailed herein.

In an embodiment, the device is configured to provide data that is configured to be used as, and/or is, output data. The output data can be a simple language description of the likely range of performance to be expected by the person after receiving the sensory aid and using the sensory aid. Also, in some embodiments, the output data is a simple language description of a chance of achieving a clinically significant improvement by person after receiving the sensory aid and using the sensory aid and/or the output data includes information regarding prescribed activities that the hearing impaired person should and/or must engage in to achieve the prediction performance. In some embodiments, the output data includes information regarding functional activities in which the hearing impaired person is likely to be able to subjectively satisfactorily engage.

In an embodiment including the cohort comparator, the cohort comparator automatically identifies a bespoke cohort of people with statistically significantly similar and/or the same pre-sensory aid use factors as the sensory impaired person, the device is configured to, based on the automatically identified bespoke cohort of people and based on the input into the input suite, automatically predict performance capabilities of the sensory impaired person after receiving a sensory aid and using such for a given period of time. This could be done using a model and/or artificial intelligence and/or the results of and/or product of machine learning. This could be done with another type of algorithm. In an embodiment, the performance capabilities include magnitude and/or range of outcomes of performance on one or more types of speech tests.

In an embodiment of the device above, the sensory aid is a cochlear implant, the sensory impaired person is a hearing impaired person, the performance capabilities include hearing related performance capabilities of at least one or more of: within 1 week from switch-on, within four weeks of 3 months after switch-on, within four weeks of 6 months after switch-on, or within four weeks of 12 months after switch-on and/or within X weeks from Y months from switch on, wherein X is 1, 2, 3, 4, 5, 6, 7, 8, 9, 10, 11, 12, 13, 14, 14, 16, 17, 18, 19 or 20 or any value or rage of values therebetween in 1 increment, and Y is 1, 2, 3, 4, 5, 6, 7, 8, 9, 10, 11, 12, 13, 14, 14, 16, 17, 18, 19, 20, 21, 22, 23, 24, 25, 30, 35, 40, 45, 50, 55 or 60, 80, 100, 125, 150, 170 or 200 or any value or rage of values therebetween in 1 increment.

In an embodiment, again where the sensory aid is a cochlear implant, the sensory impaired person is a hearing impaired person and the performance capabilities include performance on one or more types of speech tests, the performance being a comparison between preoperative performance and postoperative performance, the postoperative performance being within X weeks from Y months from switch on, wherein X is 1, 2, 3, 4, 5, 6, 7, 8, 9, 10, 11, 12, 13, 14, 14, 16, 17, 18, 19, or 20, or any value or rage of values therebetween in 1 increment, and Y is 1, 2, 3, 4, 5, 6, 7, 8, 9, 10, 11, 12, 13, 14, 14, 16, 17, 18, 19, 20, 21, 22, 23, 24, 25, 30, 35, 40, 45, 50, 55 or 60, 80, 100, 125, 150, 170, or 200, or any value or rage of values therebetween in 1 increment.

In an embodiment where the electronics includes the cohort comparator, the cohort comparator includes at least five digits of subjects' records (e.g., at least 10,000, 15,000, 20,000, 25,000, 30,000, 35,000, 40,000, 50,000, 75,000, or 100,000, or more or any value or range of values in 1,000 increments). Again, where the electronics includes the cohort comparator, the cohort comparator is configured to automatically identify a cohort based on the received input using filters that identify and/or isolate a cohort of subjects similar (including statistically similar, including substantially statistically similar) to the sensory impaired person. By way of example only and not by way of limitation, the ages 53 and 55 would not be too dissimilar between each other (and thus the cohort of age 55 would be applied against the person of age 53), whereas the ages of 20 and 55 would be dissimilar, and those cohorts would not be applied to each other. Similar could be where the different outputs of the prediction model could be within the test-retest variance of the measure of performance.

In an embodiment, again where the electronics includes the cohort comparator, and the sensory aid is a cochlear implant, the cohort comparator is configured to automatically retrieve postoperative clinical outcome data and/or functional outcome data for one or more historical subjects stored in the electronics that have a nexus with the sensory impaired person's preoperative clinical measures and functional ability and predicted performance capabilities corresponds to at least some of the automatically retrieved postoperative clinical outcome data and/or functional outcome data.

Embodiments include trajectory of performance evaluation and/or predictions. Sensory devices, such as cochlear implants, can have recipients thereof where their postoperative/post beginning of use performance changes overtime during habilitation or rehabilitation. Typically, recipients' postoperative outcomes improve from switch-on/beginning of use of the sensory device. Embodiments include predicting a candidate's trajectory of performance and displaying the trajectory to the candidate. In an embodiment, this can aid in setting of expectations and performative objectives. Large datasets, post-operative clinical outcomes measures collected from beginning of use of the sensory device, such as from switch-on and towards perpetuity, can be stratified and aggregated to form categories of trajectories of performance. These categories pertain to different levels of performance from low to high, qualified and/or quantified. A probabilistic classifier can use preoperative factors about the candidate and potential post-beginning of use, postoperative factors to select the performance trajectory category of the highest likelihood to occur. The trajectory of performance selected is presented, automatically in some embodiments, with descriptions of the stages of performance increases, the potential functional outcomes gains and the performative activities in which the candidate should engage.

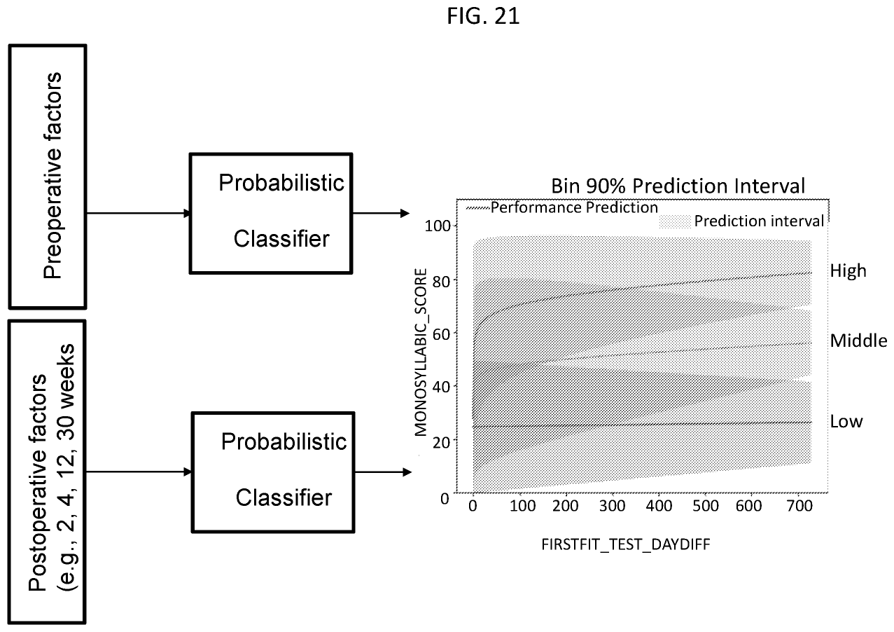

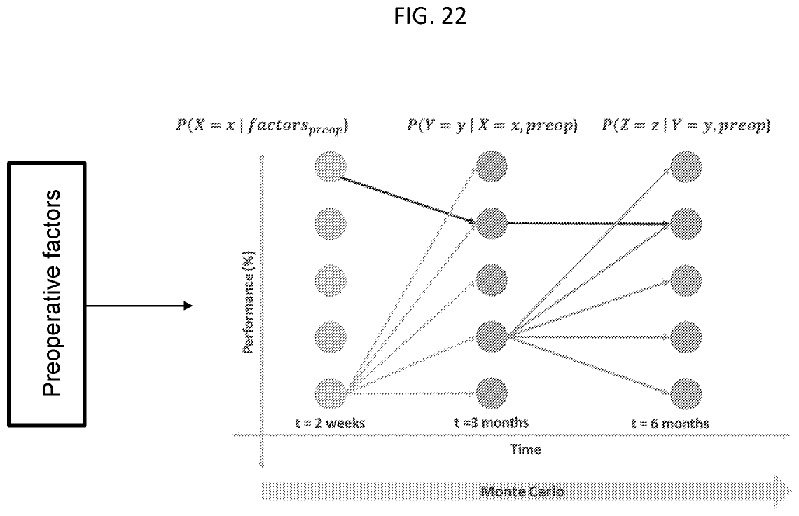

FIG. 21 shows an exemplary functional schematic of a trajectory of performance classifier.

In view of the above, with respect to methods 600 and 700, the prediction can be a prediction of a trajectory of performance that is likely to be expected. In an embodiment, the trajectory goes out for less than, greater than and/or equal to Z days where Z equals 45, 60, 75, 90, 120, 150, 200, 250, 300, 350, 400, 450, 500, 600, 700, 800, 900, 1000, 1250, 1500, 1750, 2000, 2500, or 3000 days or more, or any value or range of values therebetween in 1 day increments. In an embodiment, the trajectory provides details in A weeks either side of Z days from the beginning of use of the sensory device (switch on for example), where A equals 1, 2, 3, 4, 5, 6, 7, 8, 9, 10, 11, 12, 13, 14, 14, 16, 17, 18, 19, 20, 21, 22, 23, 24, 25, 26, 27, 28, 29, or 30, or any value or rage of values therebetween in 1 increment.

And with respect to method 800, the scenarios include performance changes over time linked to performative activities the person should engage in to achieve the changes. By way of example only and not by way of limitation, after six months from first switch on of a cochlear implant for example the recipient of the cochlear implant should be able to have a discussion with another person in a quiet environment without the use of a text aid machine, and to achieve this, the person should have utilized the cochlear implant in conversations for at least 150 hours, cumulative, since switch on, based on his or her imported data. Another example is that after a year from first switch on of the cochlear implant for example, the recipient of the cochlear implant should be able to recognize the difference between a male and a female's voice 80% of the time, and to achieve such, the recipient should have engaged in conversations where at least 30% to 40% of the conversations with a person of an opposite sex from the remainder of the conversations.

Corollary to this is that in an exemplary embodiment, the scenarios of method 800 can include performance expectations using the sensory aid after dedicated practice with the sensory aid by the person. And the dedicated practice can be qualified and/or quantified. Indeed, the candidate can be given a practice book and/or otherwise provided access to a practice regime, over the Internet or on a smart phone as an application, etc., and notified that if the candidate follows the practice regime, the outcome scenarios should likely result. And consistent with the teachings above, percentage likelihoods of achieving those scenarios with that practice can be provided.

And with respect to the device detailed above, in an embodiment, the device includes a probabilistic classifier that uses the input into the input suite to identify a performance trajectory category of the sensory impaired person after receiving the sensory aid, the performance trajectory category corresponding to the predicted performance capabilities.

And referring to the system detailed above, the artificial intelligence subsystem is configured to, based on the input, determine potential functional outcomes over time for the person using the hearing device. For example, at 3 months after switch on for a cochlear implant, the person should be able to do XYZ, and at 6 months after switch on, that person should be able to do ABC (providing that the person does what he/she should do, as would be conveyed to the person).

Still further with respect to method 1800, a variation of that method can include the action of developing a cohort model based on the obtained data, consistent with the teachings above. In an embodiment of this variation of the method, the action of analyzing the obtained data results in the development of a cohort model based on the obtained data, and the predictive algorithm uses the cohort model in a brute force manner (as opposed to an artificial intelligence based manner-here, the algorithm sifts through the data to find, for example, the closest subject(s) to the sensory impaired person to then use the data on the closest subject(s)) to predict sensory performance based on input specific to the sensory impaired person who is not one of the individuals.

A modeling approach that can be used in some embodiments is the use of a tailored Monte Carlo algorithm. FIG. 23 provides an exemplary conceptual schematic. The Monte Carlo algorithm is structured as nodes and edges. At various points in time (layers) post-operatively at sets of nodes. The sets of nodes in a layer are stratified by the magnitude of the clinical measure. The edges connect nodes to nodes of a previous layer. The transitions between nodes of a previous layer to a subsequent layer is governed by a transition probability. The transition probability is conditioned by the occurrence of the current node and exogenous factors. Exogenous factors can include, for example, pre aid use/preoperative factors, time using the device, rehabilitation activities, etc. For the first layer, the probability of selecting a particular node is conditioned by the pre aid use/preoperative factors. A layer's probability mass function is established when the transition probability for each node is calculated and normalized.

In an embodiment, the Monte Carlo process is run beginning with the first layer where a node is selected at random according to the probability mass function of the first layer. Thereafter, the transitions from a node of the previous layer to a node of the subsequent layer, a node of the subsequent layer is selected at random according to the probability mass function of the subsequent layer. For an iteration of the algorithm, the selection of nodes throughout the layers is a trajectory. The algorithm is run many times. The set of trajectories establishes a distribution describing how the candidate's performance will change over time from sensory aid beginning of use/switch-on.

In an embodiment, during the counselling conversation, interventions can be made at certain points in time (different layers of the algorithm) which changes the conditioning of the calculation of the transition probabilities. Interventions can comprise changing the degree of device usage, rehabilitation activities and/or altering other aspects of the candidate's hypothetical behavior.

Thus, with respect to method 800 for example, the data obtained pertaining to the person includes two distinct types, a first type that corresponds to demographic data, and a second type that corresponds to actions that the person declares he/she will take while using the given sensory aid. In this regard, in an exemplary embodiment, the hearing impaired person can be queried as to what he or she intends to do after obtaining the hearing device, or otherwise what he or she is willing to do after obtaining the hearing device. This can be interactive or can be based on a static set of questions. Further, in an embodiment, method 800 further includes the action of conveying to the person the developed data, wherein the conveyed developed data includes information indicative of a more favorable outcome with the use of the given sensory aid than another outcome based on the second type of data relative to what would be included in the second type of data if the person declared another action that he/she will take while using the given sensory aid. And corollary to the above, in an embodiment, the software includes a Monte Carlo algorithm that relies on the first type of data to start a Monte Carlo process and the second type of data when intervening at certain points in the process.

In an embodiment, the second type of data includes at least a first action declared by the person and a second action contradictory to the first action declared by the person. For example, the person can declare that he or she will not practice or otherwise not use cochlear implant “drills,” and that can be the first action, and the second action can be that the person will participate or otherwise utilize cochlear implant “drills” at least three times a week for at least an hour each time. The first action could be that the person will engage in one conversation per day with the same person (e.g., his or her spouse), and the second action could be that the person will engage in at least three conversations per day with three different people. A wide variety of different actions can be declared. The idea is that certain actions by the candidate or otherwise the prospective recipient of the cochlear implant for example will be more conducive to obtaining better efficacious results over the time periods detailed herein than other actions or other activities.

In an embodiment, the method further includes conveying to the person the determined data, wherein the determined data includes first information indicative of a first outcome with the use of the given sensory aid based on the first action and second information indicative of a second outcome with the use of the given sensory aid based on the second action. This can correspond to qualitative information and/or quantitative information that will be different for the different trajectories, or for the various ranges for a given likely trajectory. For example, the functional outcomes could be more efficacious for the second actions than the first actions. Scores on clinical measures could be higher or otherwise better for the second actions then for the first actions. And the more that the hearing impaired person practices or otherwise utilizes the cochlear implant, the better the progression (higher efficacy/better results and/or faster movement along the trajectory) along the trajectory relative to that which would otherwise be the case.

In view of this, in an embodiment, the different outcomes that will result from different actions that the candidate is willing to execute can be presented to the candidate, to show the candidate the possible outcomes if he or she “tries harder.” Alternatively, this can address unrealistic expectations. In this regard, the person that believes that the cochlear implant will be a panacea and that he or she need only undergo the surgery and obtain the prosthesis and will hear perfectly and understand all speech within days of switch on can be dissuaded of such a view. Accordingly, the candidate will understand that he or she must partake in certain activities and otherwise do certain things in order to obtain an increased efficacious results of the cochlear implant. And this can have an impact on encouraging the candidate, after becoming a recipient of the cochlear implant, to engage in these activities. Put another way, the plan for habilitation and/or rehabilitation can be put in place, at least partially, or at least embryonically, prior to the surgery, and this way the psychological impact of going into the process with a plan and otherwise having more realistic expectations should improve the outcomes, which improvements should be able to be measured with clinical measures.

In any event, the various teachings detailed herein can provide a trajectory of performance prediction that can have utilitarian value. This can be useful to not just determine the performance level where the candidate is likely or otherwise might achieve utilizing a sensory prostheses, but also to show the level of performance where the candidate might be in the early days after first utilizing the sensory prostheses. By setting expectations at a realistic level, it is more likely that the candidate will continue his or her efforts to practice with the prostheses or otherwise expend effort to improve the performance that results from the prostheses. This is opposed to a scenario where the candidate believes that the prostheses will provide a certain level of performance at the early stages and then be disappointed and otherwise discouraged when the prostheses provides a lower level of performance below his or her expectations, however unreasonable those expectations might have been. Corollary to this is that if indeed the candidate is a candidate where the trajectory of performance will be relatively flat (the performance level in the early days will be about the same as that a year or two or three out), this too will avoid discouraging the candidate after he or she begins use of the prostheses because the candidate will know that the performance level is not likely to improve. In essence, the trajectory of performance features of the teachings herein provide expectations management which can be usually important with respect to compliance and otherwise achieving higher efficacy results utilizing a cochlear implant, and often can manage expectations sufficiently so that the recipients of the implant will not quit utilizing the implant after a few weeks or few months because the performance is not what he or she expected including the trajectory of the performance.

Embodiments also include devices and systems that have a risk of delay of treatment prediction function, and also include methods of predicting the risk of delay of the treatment. In this regard, by way of example only and not by way of limitation, there are many causes of hearing loss, the most common causes are general noise exposure and presbycusis. Sensory loss, such as hearing loss, negatively impacts the quality of life of those impacted. In addition to the primary causes of the person's hearing loss, there may be negative reinforcement of hearing loss due to lack of stimulation of the auditory neural pathways and processing centers known as neural atrophy. Analogous effects can be seen with respect to the other sensors, such as balance and vision by way of example. As such, there are risks associated with delaying the treatment of sensory loss, such as for example hearing loss, such as the increase in severity of sensory (e.g., hearing) loss and loss in quality of life improvements.

Embodiments include a risk of delay model that takes the current, or from multiple instances in time, preoperative factors about a person afflicted with sensory loss, such as hearing loss or who will experience sensory loss, such as hearing loss, such as age, gender, aetiology, questionnaires and clinical measures of sensory (e.g., hearing, balance, vision) loss and projects the further progression of sensory (e.g., hearing, balance, vision) loss in the form of clinical measures. The projected progression of sensory (e.g., hearing, balance, vision) loss presents the associated clinical measures at regular time intervals from the current point in time (any of the time periods detailed herein) and/or a point in time in the future (e.g., 1, 2, 3, 4, 5, 6, 7, 8, 9, 10, 11, 12, 13, 14, 15, 16, 17, 18, 19, 20, 25, 30, 35, or 40 or more, or any value or range of values in 1 increment weeks or months). Functional activities that the person typically engages in at different magnitudes of clinical measures are additionally associated in some embodiments. Each prospective time point is compared against the current state and the model presents the expected decline in clinical measures, functional activities and/or quality of life. This is information is processed into a language form to be used during counselling of the candidate/patient.

The rates and nature of the progression of hearing loss or vision loss or balance loss, or other types of sensory loss, differs based on the cause, severity and duration of the sensory loss. Using large datasets of subjects' demographics, health and hearing health data, and history of preoperative/pre hearing device use and postoperative/post beginning of the sensory device/post beginning of the treatment (note that while this exemplary embodiment has been focused on sensory loss, this concept is applicable to non-sensory negative medical conditions as well and thus any disclosure herein regarding a treatment utilizing a sensory supplement device corresponds to a disclosure of utilizing some other medical device for a nonsensory related negative medical condition and/or a treatment that does not necessarily involve a medical device per se for a negative medical condition) use clinical measures, unsupervised clustering algorithms can identify groups of subjects with similar patterns of progression of the sensory loss. For each group, key factors are identified to form a signature of the group. Each group has a characteristic hearing loss pattern over time. When a candidate's factors are inputted into the model, the group that is the closest match is selected, and the characteristic sensory loss pattern is adjusted in accordance with the candidate's/patient's current and/or historical clinical measures. The adjusted characterized sensory (hearing or vision or balance, etc.) loss pattern is the projected sensory loss progression. Thus, embodiments can utilize a prediction model to develop data upon which to predict the risk of delay and/or can utilize a cohort comparator to identify a cohort of people with statistically significantly similar and/or the same pre-sensory aid use factors as the sensory impaired person and develop a risk of delay forecast based on the identified cohort, more accurately, the information associated with the identified cohort in the records of the database for the members of that cohort.

In view of this, returning back to methods 600 and 700, the application of a sensory supplement device is the lack of use of the sensory supplement device relative to the use of the sensory supplement device over a given time period, where this time can be any of the time periods detailed above. Again with reference to these methods, in an exemplary embodiment, the prediction is a risk of delay of use of the sensory supplement device by the human. In an embodiment of these methods, the prediction indicates clinical measures, functional activities and/or quality of life of the human at various times in the future with and/or without use of the sensory supplement device by the human. In an embodiment, the prediction indicates the difference in clinical measures with and without the use of the sensory supplement device by the human. And again, while this embodiment is focused on a sensory supplement device, this corresponds to a disclosure for the purposes of textual economy of a prediction associated with medical devices that are not related to treating sensory loss and/or other types of treatment.

In an embodiment of method 800, the database includes categories of trajectories of performance for habilitation and/or rehabilitation for the statistically significant number of individuals and the method further includes identifying the most likely category based on the obtained data. In an embodiment, a percentage range associated with one or more trajectories can be identified. This can have utilitarian value with respect to showing the candidate for a treatment the different trajectories that could exist and the percentage of probability that the person will fall into those trajectories.

It is briefly noted that in some embodiments, any disclosure of developing or identifying information or data etc., corresponds to an action of ultimately conveying such data to the healthcare professional and/or the candidate in raw format or in a translated and/or a natural language manner.

Embodiments include devices systems and/or methods for and of translating clinical measures into functional outcomes. In this regard, many of the models and/or the cohort comparator based teachings herein provide results in clinical measures. Scores on sound tests or speech tests, etc. These will be far more valuable or otherwise useful to an audiologist or a professional than a hearing impaired person of average knowledge and/or average experience, or even above average knowledge including considerably above average knowledge and/or above average experience. The audiologist and/or otherwise healthcare professional who has training and/or experience in the given technology will find the clinical measures more meaningful. Embodiments thus can include the translation of these clinical measures into functional outcomes.

More specifically, clinical measures are standardized measures of for example, hearing performance or loss. These measures can, in some scenarios, directly translate to the same magnitude and/or frequency of functional activities across subjects. A clinical measures to functional outcomes translation engine that exists in some embodiments automatically translates the type and magnitude of a clinical measure (or set of clinical measures) to a probability of engaging in a particular functional activity or a set of functional activities. This translation engine's accuracy can be optionally improved and personalised by taking into account demographic and aetiology data about the subject. Using large datasets of subjects' demographic, health and hearing health data and subjects' notifications of the types, magnitude and/or frequency of functional activities they engage, conditional probabilistic relationships can be established between clinical measures, exogenous variables and functional activities. The equation below is an exemplary equation that captures that general form of this which states that the probability of engaging in an activity is condition on one or more clinical measures and other exogenous factors. Additional approaches for calculating the probability of engaging in activities include the use of Bayes Theorem and Naïve Bayes Classifier, and the engine/computer algorithm, etc., can utilize any of these:

P(activity)=P(activity|clinicalₘₑₐₛᵤᵣₑ,exogenous)+ε

The outcomes prediction, trajectory of perform and risk of delay models and/or algorithms can, in some embodiments, integrate the translation engine or algorithm, etc. to transform the outputs of the models into probabilities of engaging in functional activities. There can be further translation of these outputs into language that is meaningful and actionable to clinicians and/or the candidates (automatically for example).

Functional activities and/or outcomes may include any of those detailed herein. In an embodiment, the artificial intelligence subsystem(s) and/or another subsystem of the overall system(s) detailed herein can be configured to translate a type and magnitude of the clinical measure(s) to a probability of the person engaging in a particular functional activity and/or a set of functional activities. For example, there will be at least a 75% chance that the person will be able to listen to a standard brief weather report and/or traffic report on WMAL or WTOP at or around 5 p.m. in Washington DC. For example, there will be between a 50 and 60% chance that the person will be able to determine a direction of a standard car horn sound, for example, there will be no more than a one in three chance that the person will be able to engage in a conversation in a noisy environment, and the noise can be qualified and/or quantified. In an embodiment, there is an expanded version of method 1700, where the software includes conditional probabilistic relationships between clinical measures, exogenous variables and functional activities in which the person can engage. For example, a cconditional probability is the probability that a value of X occurring given that we know the particular value of Y P(X=x|Y=y), where, applied to hearing P(X=x| age, aetiology, preoperative performance), one would then iterate through each value of X to form a probability mass function.

And in a variation of the method 800, the obtained data includes the sensory health data and notifications of type, magnitude and frequency of functional activities of which the individuals engage, the action of analyzing the obtained data includes establishing probabilistic relationships between clinical measures, exogenous variables and functional activities of the obtained data and the predictive algorithm includes the established probabilistic relationships. With respect to the devices and/or systems detailed above, in an embodiment, the electronics is configured to transform results of the prediction model into probabilities of the person of engaging in functional activities and/or the electronics is configured to transform results of the cohort comparator into probabilities of the person of engaging in functional activities. Further, with respect to the devices and/or systems detailed above, the electronics can be configured to transform results of the prediction model into language that is meaningful and actionable to the mean clinician and/or the electronics is configured to transform results of the cohort comparator into language that is meaningful and actionable to the mean clinician. In an embodiment, the mean clinician is the 50 percentile clinician in the field of the sensory aid at issue. For example, if the sensory aid is a cochlear implant, it would be the mean clinician in the field of cochlear implants, and this would be different than, for example, the mean clinician in the field of conventional hearing aids for example. In an exemplary embodiment, such as where the cohort comparator brings back a certain cohort, the devices and/or systems can be configured to analyze that cohort and provide a prediction based on that certain cohort, in this prediction is what is put into the language that is meaningful and actionable to the mean clinician in the field of the sensory aid. Conversely, for example, the certain cohort is what is transformed (e.g., language detailing the cohort in qualitative and/or quantitative terms).

With regard to the system detailed herein, in an embodiment, the input regarding information about the person includes clinical measure(s) of hearing performance and/or loss. In an embodiment, of the system, the input regarding information about the person includes demographic and/or aetiology data about the person and the system is configured to translate a type and magnitude of the clinical measure(s) and the demographic and/or aetiology data to a probability of the person engaging in a particular functional activity and/or a set of functional activities.

Embodiments also include expectations management implementations. Potential cochlear implant candidates and candidates have a range of different expectations about the efficacy and/or quality of life improvements that can be obtained/achieved by the implementation of cochlear implant system to treat hearing loss. This is also the case with respect to candidates and potential candidates for conventional hearing aids and the various types of bone conduction devices and retinal implants etc. this is also the case for different types of treatment for other nonsensory related conditions. When expectations are not in line with what could be achievable, it may cause recipient of these medical devices or people that undergo certain treatments for a negative medical condition to regret undergoing treatment, and this can negatively impact behavior. The expectation management feature can facilitate the clinician to run a tailored conversation (a bespoke conversation) with the candidate or potential candidate (herein, any use of candidate corresponds to an alternate of disclosure of a potential candidate and vice versa unless otherwise noted, providing that the art enables such a candidate is someone who meets an established indication for cochlear implantation, whereas a potential candidate is someone who has not yet fully evaluated (or had fully evaluated) the facts to see if they meet the indication for cochlear implantation, regarding expectations of the treatment and what is expected if the patient in terms of performative behavior. In an embodiment, to have candidates progress to undergoing treatment can include demonstrating how well that person would perform with a cochlear implant, which can be achieved using the teachings herein for example. As noted above than a brief manner, this can have utilitarian value with respect to avoiding discouragement scenarios which discouragement scenarios can result in the recipient of the medical device or the person undergoing the treatment to not try as hard and in some instances, give up, which could entail halting the treatment or not using the cochlear implant (just leaving the external component in the desk drawer every day for example). The latter results in an effective efficacy of zero for that cochlear implant.

In an embodiment, the inputted data into the system/device/the acquired data/obtained data can be:
Candidate preoperative factors such as demographics, aetiology, hearing history, status of language development, etc.
The outputs (and corresponding probabilities of functional activities) of the outcomes prediction, trajectory of performance and/or risk of delay models/algorithms
Questionnaire responses of the candidate's beliefs regarding the efficacy and benefits of sensory supplement device, such as the cochlear implant, treatment counselling

In some embodiments, algorithms compare the candidates' beliefs about treatments (e.g., cochlear implant implantation, retinal prosthesis implantation, etc.) against normative efficacy data and the personalised efficacy predictions for the patient. The systems and devices can be configured to determine and the methods can include, if the comparator algorithms identify a substantial difference between the candidate's beliefs and normative efficacy data or personalised efficacy predictions, these differences can be raised to the user of the application (e.g., the clinician or medical professional or healthcare professional, or the candidate if the system is utilized in a self-directed application). The information raised to the user can include the details of the difference between the patient's beliefs, such as, for example:
The type of treatment that the sensory supplement device, such as a cochlear implant or a retinal implant or a bone conduction device, provides
The extent of sensory performance, such as hearing performance, that is achievable and further curation of this based on personalised details
The extent of functional activities that are achievable and further curation of this based on personalised details
The performative activities that the candidate should engage in to achieve the expected sensory performance, such as hearing performance, and/or functional activities
Notification of whether the candidate is likely to adhere to prescribed to performative activities (e.g., candidates with poor locus of control are unlikely to adhere to prescribed to performative activities)

In an embodiment, the user can select the most pertinent differences for personalised counselling on the treatment.

As noted above, a portion of cochlear implant candidates, including a proportion of cochlear implantation recipients, discontinue the treatment after having undergone the process of preoperative consultation, surgery, switch-on and/or post-operative counselling. Machine learning and/or probabilistic models can be developed using utilitarian datasets of expectations indicators, psychological factors and/or postoperative device usage logs to estimate the likelihood or probability that a candidate will discontinue use of the sensory system, such as a cochlear implant system. When the user is using the personalized counselling application, during the expectations management session, the candidate's expectation indicators and/or psychological factors can be solicited, such as via conversion and/or questionnaires, and these solicited indicators can be inputted into the system/device or otherwise used in various methods. For example, the machine learning or probabilistic model can be run based on this information and the probability or likelihood of discontinuation of the use of the sensory device, such as discontinuation of the use of the cochlear implant, is raised to the user. If the user is a clinician, the user can use this information to counsel the candidate about the importance of continued usage of the sensory device post acquisition (such as continued use of the cochlear implant post-implantation) and/or allocate appropriate resources to postoperative counselling and care.

In view of this, in an embodiment, an expanded method 600 or 700 can include the action of obtaining data relating to beliefs of the person regarding future efficacy of the sensory supplement device for that person and/or future benefits of the sensory supplement device for that person. The methods further include, based on the obtained data relating to the at least demographic and sensory performance of the human and the obtained data relating to beliefs of the person, determining at least one of whether the person exposes a realistic belief regarding the future efficacy of the sensory supplement device for that person and/or future benefits of the sensory supplement device for that person or a deviation, qualitatively and/or quantitatively, of the person's beliefs from a statistically supported likely outcome regarding the efficacy of the sensory supplement device for that person and/or benefits of the sensory supplement device for that person. Methods can include an expanded method 600 or 700 can include the action of developing, using a probabilistic model and/or results of artificial intelligence, a prediction of whether and/or a percentage chance that the human will discontinue use of the sensory supplement device after beginning to use such based on the obtained data, and in an embodiment, the discontinued use can occur within 6, 9, 12, 15, 20, 25, or 30 months of beginning use and the person is expected to live for at least twice those values.

Embodiments include devices and/or systems where the electronics includes a product of artificial intelligence and/or a probabilistic model that estimates a likelihood and/or probability that the person will discontinue use of the sensory aid.

Referring to method 1700, in an embodiment, there is an expanded method that includes obtaining data relating to beliefs of the person regarding future efficacy of the sensory aid for that person and/or future benefits of the sensory aid for that person and comparing (e.g., automatically), using a computer chip or a logic circuit or electronics or software, the developed data to the beliefs of the person to develop a quantitative and/or qualitative assessment of how closely the person's beliefs are to statistically likely outcomes. In an embodiment, the data obtained relating to beliefs of the person can correspond to natural language beliefs, and a clinician or the like or the systems and/or devices disclosed herein can transform that into a range of clinical measures, such as scores on standardized speech tests or the like that have a correlation to the patient's beliefs, and these clinical measures can be compared to the actual expected clinical measures to assess how closely the person's beliefs are to the statistically likely outcomes. In an embodiment, the transformation into clinical measures is not done or otherwise this evaluation can be performed without doing so, depending on the sophistication of the underlying system. In an embodiment, the assessment can be in general terms, such as it is reasonable to expect such results or otherwise believe certain things can happen to that is possible, to that might be an overly optimistic assessment to that is very unlikely to occur/result. In an embodiment, the assessment can be more specific, such as with percentages, etc. in an embodiment, the assessment can be more specific, such as laying out what the candidate will and will not be able to do, with, in some embodiments, the likelihood of such.

In an embodiment, there is a system as described herein, wherein one or more of: the input regarding information about the hearing impaired person includes the person's beliefs about the future efficacy of the hearing device; the system is configured to compare, using a computer chip or a logic circuit or electronics or software, the beliefs to normative hearing device efficacy data and/or personalized hearing device efficacy predictions based on the prediction, and the system is configured to provide a qualitative; and/or quantitative information regarding the comparison. The system can also be configured to, based on the person's beliefs, present information including one or more of: a type of treatment that the hearing device provides, targeted to changing the person's beliefs; an extent of hearing performance that is achievable and further curation of hearing based on the information about the hearing impaired person, targeted to changing the person's beliefs; an extent of functional activities that are achievable and further curation of functional activities based on the information about the hearing impaired person, targeted to changing the person's beliefs; performative activities that the subject should engage in to achieve the expected hearing performance and functional activities, targeted to changing the person's beliefs; or an assessment, qualitative and/or qualitative, of the likelihood that the person will adhere to prescribed and/or performative activities.

The utilization of machine learning specifically, and the artificial intelligence systems in general, enable automated or semiautomated improvement of the models disclosed herein. In an exemplary embodiment, as datasets or databases of recipients of sensory supplement devices, such as cochlear implants preuse (preoperative in the case of a cochlear implant) and post beginning of use (postoperative) factors increase in size, algorithms can be updated/revised to provide more expansive and/or more utilitarian coverage of the candidate population and/or improve precision and/or accuracy of the existing models. Embodiments thus include continuously or periodically or randomly collecting additional data to further expand the statistically significant numbers upon which the models are based or otherwise the models access. Embodiments further include retraining or providing additional training of the already trained neural network or otherwise establishing a new neural network based on new training utilizing this additional data (and also the older data if the older data is still acceptable or otherwise deemed to be utilitarian). For example, in a variation of method 800, there is an expanded version, which includes the additional action of, subsequent to the action of obtaining, obtaining additional (i) habilitation and/or rehabilitation data and/or sensory health data and (ii) demographic data for additional sensory impaired persons. This expanded method further includes analyzing the obtained additional data, separately and/or along with the obtained data, to:
(i) develop a new, second predictive algorithm for human sensory performance based on the results of the analysis of the obtained additional data, wherein the second predictive algorithm predicts performance based on input specific to a sensory impaired person who is not one of the individuals; or
(ii) update the predictive algorithm based on the results of the analysis of the obtained additional data.

This can result in a new trained neural network or a re-trained neural network. Further, the aforementioned method actions can be repeated at least 1, 2, 3, 4, 5, 6, 7, 8, 9, 10, 11, 12, 13, 14, 15, 16, 17, 18, 19, 20, 25, 30, 35, 40, 45, 50, 75, 100, 125, 250, 175 or 200 or more times or any value or range values therebetween in one increment over a period of at least 1, 2, 3, 4, 5, 6, 7, 8, 9, 10, 11, 12, 13, 14, 15, 16, 17, 18, 19, 20, 25, 30, 35, 40, 45, 50, 75, 100, 125, 250, 175 or 200 months or any value or range of values therebetween in one week increments.

In an embodiment, at regular time intervals or irregular time intervals, the model/algorithm training algorithm can engage in one or more of:
Segmenting new subject data into model development and validation datasets
Validating the precision and/or accuracy of the existing iterations of model/algorithms
Validating comparisons subject's expectations to personalised model/algorithm expectations
Further updating of the coefficients, weights and thresholds of models/algorithms using the updated model development dataset
Revalidation of updated models/algorithms on one or more validation sets to discern whether improvements have been achieved
Deployment of updated models and algorithms/new models/algorithms to the end user applications

Thus, in an even further expanded method 800, the expanded method thus detailed can thus further include segmenting new data into model development and validation datasets; validating precision and accuracy of the existing algorithm; validating comparisons subject's expectations to personalized model/algorithm expectations and revalidation of the updated algorithm and/or revalidation of the new second algorithm on one or more validation sets to discern whether improvements have been achieved.

And note that embodiments can include withholding the deployment of the updated models and/or algorithms. In this regard, as noted above, in some embodiments, the models and algorithms are stored and otherwise maintained at a central location, such as a location under the control of the medical device manufacturer or an entity under the control of such for example. Thus, the models would not necessarily be deployed to the end user applications. Instead, the end user applications would be applications that can access the models such as via the Internet or the like or over a landline or a cell phone link. The data provided from the end user can be processed at the remote location, and the results sent back to the end user. The remote nature of the processing could be transparent to the end user.

Corollary to this is that in an embodiment, the devices and/or systems disclosed herein can have access to an internet connected platform comprising data storage and/or model training algorithms. Users (clinicians and/or candidates) can provide their consent to:
Having their data inputted to be transmitted to the cloud platform
Having details about their cochlear implantation surgery (or whatever applicable surgery) uploaded
Having their postoperative hearing performance, quality of life and participation in functional activities monitored for a specified time periods
Extent of pseudonymization

As clinicians and patients consent for their preoperative factors and postoperative factors, in the example of a cochlea implant, to be uploaded to cloud storage system, the volume of data increases. The record of consent is also uploaded to the data storage of the cloud platform. The idea here is that the additional data can be passively gathered on an ongoing basis for example for a periodic basis for example if end users actively upload the new data to the database where the data is maintained and/or if the end users permit their information to be periodically or continuously “swept” over the Internet to the database. This additional data can be added to the existing data or otherwise utilize for retraining purposes or for training a new network.

It is briefly noted that in at least some exemplary embodiments, the various information developed by the teachings detailed herein is provided to the clinician and/or the candidate directly or in a modified form including a translated form by way of example. The ultimate goal in at least some exemplary embodiments is to utilize the teachings detailed herein for the purposes of counseling a person who suffers from a negative medical condition with respect to treatment options and the likely results of those options. The idea is to persuade the person to engage in a treatment option that could have utilitarian value, including treatment options that the person is hesitant to adopt, such as for example the treatment option of a cochlear implant instead of a conventional hearing aid having diminished efficacy because of continued hearing loss progression.

As can be seen above, many examples have been presented in terms of a hearing prosthesis in general, and a cochlear implant in particular. Phrases such as preoperative and postoperative have been utilized herein. These are but exemplary embodiments, and it is noted that any disclosure herein of a cochlear implant and a preoperative and a postoperative state corresponds to a disclosure of an alternate embodiment of another device and/or another treatment where the preoperative and postoperative dates correspond to the time before use of the device and the beginning of use of the device respectively. Also, as noted above, embodiments include utilizing the teachings detailed herein with a treatment does not specifically require the use of a medical device for the treatment. Accordingly, any disclosure herein to the preoperative and postoperative dates/pre beginning of use dates and post beginning of use dates corresponds to a disclosure of pretreatment and the beginning of treatment respectively, and the disclosure of the device utilized in the treatment corresponds to a disclosure of the treatment.

Embodiments can include a system and/or simply an embodiment that includes a non-transitory computer readable medium having recorded thereon, a computer program for executing at least a portion of a method, the computer program including code for executing any one or more of the method actions and/or functionalities detailed herein. Thus, any disclosure herein of a method action or functionality corresponds to a disclosure of a non-transitory computer readable medium having programed thereon code to execute one or more of those actions and also a product to execute one or more of those actions.

Embodiments include any functionality disclosed herein and/or method action disclosed herein being executed by a computer chip, a processor, software, logic circuitry and/or electronics, and all are not mutually exclusive. Any circuit that can enable the teachings herein can be used providing that the art enables such. Thus, in the interests of textual economy, and disclosure herein of a functionality of an article of manufacture corresponds to any one or more of the aforementioned structures being configured to execute such and otherwise for such, and the same is for any method action disclosed herein, where any such action corresponds to a disclosure of any one or more of the aforementioned structures being configured to execute such and otherwise for such.

In some embodiments, a neural network, such as a DNN, is used to directly interface to the input into the systems/devices detailed above, and process this input via its neural net, and determine the information detailed above. The network can be, in some embodiments, either a standard pre-trained network where weights have been previously determined (e.g., optimized) and loaded onto the network, or alternatively, the network can be initially a standard network, but is then trained to improve specific recipient results based on outcome oriented reinforcement learning techniques.

Any disclosure herein of a processor corresponds to a disclosure in an embodiment of a non-processor device or a combined processor-non-processor device where the non-processor is a result of machine learning. Embodiments can include a link from the cloud to a clinic to pass information back and forth, enabling the remote processing noted above and/or enabling the obtaining of additional data for retraining purposes. Information can be uploaded to the cloud to the clinic, where the information can be analyzed. Another exemplary system includes a smart device, such as a smart phone or tablet, etc., that is running a purpose built application to implement some of the teachings detailed herein. This can be used by the clinician, and can contain at least the front end portions of the systems and devices detailed herein, or otherwise provide the interface portal to the back end. Any disclosure herein of a processor corresponds to a disclosure of a non-processing device, or includes non-processing devices, such as a chip or the like that is a result of a machine learning algorithm or machine learning system, etc.

In an exemplary embodiment, the smart device can be configured to present the windows for the interface that will be used by the user.

Reference herein is frequently made to the recipient of a hearing prosthesis. It is noted that in at least some exemplary embodiments, the teachings detailed herein can be applicable to a person who is not the recipient of a hearing prosthesis. Accordingly, for purposes of shorthand, at least some exemplary embodiments include embodiments where the disclosures herein directed to a recipient correspond to a disclosure directed towards a person who is not a recipient but instead is only hard of hearing or otherwise has a hearing ailment and is contemplating obtaining a hearing assistance device.

Any method action and/or functionality disclosed herein where the art enables such corresponds to a disclosure of a code from a machine learning algorithm and/or a code of a machine learning algorithm and/or a product of machine learning for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. In an embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code-code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

And to be clear, in an exemplary embodiment, there are products of machine learning algorithms (e.g., the code from the trained machine learning algorithm) that are included in any one or more of the systems/subsystems detailed herein, that can be utilized to analyze any of the data obtained or otherwise available disclosed above that can be utilized or otherwise is utilized to evaluate the data obtained herein. This can be embodied in software code and/or in computer chip(s) that are included in the system(s).

An exemplary system includes an exemplary device/devices that can enable the teachings detailed herein, which in at least some embodiments can utilize automation. That is, an exemplary embodiment includes executing one or more or all of the methods and/or functionalities detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

Prediction can be represented by an algorithm where circuitry receives the input (embodied in an analogue or a digital signal), where the input suite converts the “physical” input into electronic signals using analog to digital converters for example, or in the case of the input suite corresponding to an Internet server, receives the digital signal from a remote location, and the digital data is stored in a memory and/or received by the electronics. The electronics, which is a result of the machine learning, takes the digital signal and deconstructs the digital signal to evaluate properties, and then, using its “knowledge” from its training, provides an output corresponding to the prediction. By analogy, the operation is analogous to how a human being “predicts” how he or she will function if he or she foregoes a meal for example, or stays up all night, or if he or she drinks 5 cups of coffee in one hour. Past experience informs the future results, the prediction

The cohort comparator can be a database such as Microsoft™ Access, where the computer automatically matches the data instead of the human matching the data. The results of machine learning and/or a product thereof can be used to perform the automatic matching.

In an exemplary embodiment, the cohort comparator is a computer chip and/or a computer circuit. The cohort comparator can be electronics. In an exemplary embodiment, cohort comparison can be represented by an algorithm where circuitry receives the input (embodied in an analogue or a digital signal), where the input suite converts the “physical” input into electronic signals using analog to digital converters for example, or in the case of the input suite corresponding to an Internet server, receives the digital signal from a remote location, and the digital data is stored in a memory and/or received by the electronics. The electronics takes the digital data and “looks” for certain strings of zeros and ones that correspond to a match with signatures/identifiers linked to prestored data regarding performance capabilities. The data linked to the signatures/identifiers is the cohort identified.

It is further noted that any disclosure of a device and/or system detailed herein also corresponds to a disclosure of otherwise providing that device and/or system and/or utilizing that device and/or system.

It is also noted that any disclosure herein of any process of manufacturing or providing a device corresponds to a disclosure of a device and/or system that results therefrom. Is also noted that any disclosure herein of any device and/or system corresponds to a disclosure of a method of producing or otherwise providing or otherwise making such.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method, comprising:
obtaining data relating to at least demographic data and sensory performance of a human;
analyzing the data based on the obtained data using a statistical model, a probabilistic model and/or a model based on results from or that is a product of machine learning to develop output, wherein
at least one of:
the output is a prediction of results relating to application of a sensory supplement device to the human; or
the method further includes developing a prediction of results relating to application of a sensory supplement device to the human based on the output.

2. The method of claim 1, wherein:
the action of analyzing the data is executed using a code from and/or of a machine learning algorithm to develop the output.

3. The method of claim 1, wherein:
the sensory performance is hearing; and
the prediction is a magnitude of a result of a speech test and/or a change in result of a speech test.

4. The method of claim 1, wherein:
the demographic data includes age, data relating to hearing loss and speech attributes.

5. The method of claim 1, wherein:
the sensory supplement device is a cochlear implant.

6. The method of claim 1, wherein:
the action of analyzing the data based on the obtained data is executed using a model based on results from machine learning.

7. The method of claim 1, wherein:
the prediction is a prediction regarding functional activities in which the human can engage.

8-15. (canceled)

16. A method, comprising:
obtaining data pertaining to a person with a medical condition; and
analyzing the obtained data using a computer chip or a logic circuit or electronics or software to develop data regarding effects of treatment scenarios of the person if that person engages in the treatment, wherein
the computer chip or logic circuit or electronics or software is based on a statistically significant population of persons who were afflicted with the medical condition and who also engaged in the treatment.

17. The method of claim 16, wherein:
the treatment is the use of a given sensory aid; and
the computer chip or logic circuit or electronics or software is based on a statistically significant population of sensory impaired persons who obtained the given sensory aid.

18. The method of claim 17, wherein:
the computer chip or logic circuit or electronics or software is code of and/or from a machine learning algorithm; and
the machine learning algorithm is a trained system trained based on a statistically significant population of sensory impaired persons who obtained the given sensory aid.

19. The method of claim 18, wherein:
the person is a hearing impaired person; and
the sensory aid is a hearing device.

20. The method of claim 19, wherein:
the hearing device is a cochlear implant.

21. The method of claim 17, wherein:
the scenarios are based on performance of one or more United States medical community accepted sensory tests.

22. The method of claim 17, wherein:
the scenarios include qualitative values pertaining to at least two functional outcomes relating to an improved sensory experience resulting from use of the sensory aid.

23-86. (canceled)

87. A method, comprising:
obtaining assess to a database having data based on hearing habilitation and/or rehabilitation data and demographic data for a statistically significant number of individuals;
obtaining data on a hearing impaired person who is not one of the individuals;
querying the database based on the obtained data for the hearing impaired person to automatically identify a cohort of similar individuals to the hearing impaired person from among a statistically significant number of individuals; and
retrieving post hearing device usage clinical measures and/or functional outcomes for the individuals corresponding to the identified cohort.

88. The method of claim 87, further comprising:
automatically generating filters based on the obtained data for the hearing impaired person, wherein
the action of querying the database is executed based on the automatically generated filters.

89. The method of claim 87, further comprising:
based on the retrieved measures and/or functional outcomes, providing a forecast of hearing habilitation and/or rehabilitation using a type and/or design of hearing prosthesis, wherein the data of the database includes data based on habilitation and/or rehabilitation data and demographic data for a statistically significant number of individuals who use the type and/or design of hearing prosthesis.

90. The method of claim 87, wherein:
the hearing device is a cochlear implant; and
the method further comprises, based on the retrieved measures and/or functional outcomes, presenting to the person one or more of:
a range and/or distribution of postoperative clinical measures of the subjects of the cohort;
a likelihood that the person will improve by integrating a probability density function of the cohort's post operative clinical measures from where the person is currently performing to the maximum values of the clinical measures
a likelihood of different functional activities in which the person could engage if the person were to receive the cochlear implant;
relative to a date of first use of the person's hearing device, estimated dates and/or periods of time of achievement of certain ranges of clinical outcomes measures and/or functional outcomes; or
a description of how the subjects of the cohort use their hearing device.

91. The method of claim 87, further comprising:
automatically, based on the retrieved measures and/or functional outcomes, developing a plausible scenario for the individual for hearing habilitation and/or rehabilitation using a type and/or design of hearing prosthesis, wherein the data of the database includes data based on habilitation and/or rehabilitation data and demographic data for a statistically significant number of individuals who use the type and/or design of hearing prosthesis.

92. The method of claim 87, wherein:
the habilitation and/or rehabilitation data and demographic data for a statistically significant number of individuals is for individuals who have received a cochlear implant.

93-117. (canceled)
